SEQUENCE LISTING (96 sequences)

SEQ ID NO: 1 - DNA, 60 bp, Artificial
  synthetic primer
ttttgacttaactctagttcgtcatcgcatagatgatttagaaaggtcgatcagggagga

SEQ ID NO: 2 - DNA, 60 bp, Artificial
  synthetic primer
tatcggggaagaacaggttcgtcatcgcatagatgatttagaaattgcattctaaaacct

SEQ ID NO: 3 - DNA, 60 bp, Artificial
  synthetic primer
taaatacagaaaacaggttcgtcatcgcatagatgatttagaaagtcgatactatgttat

SEQ ID NO: 4 - DNA, 60 bp, Artificial
  synthetic primer
acgccaactttgaaaagttcgtcatcgcatagatgatttagaaaaagcgagctcggtact

SEQ ID NO: 5 - DNA, 60 bp, Artificial
  synthetic primer
aaaacaattcatccaggttcgtcatcgcatagatgatttagaaacggccggtagaaagga

SEQ ID NO: 6 - DNA, 40 bp, Artificial
  synthetic primer
gaacctgttcttccccgatatcctccctgatcgacctttc

SEQ ID NO: 7 - DNA, 40 bp, Artificial
  synthetic primer
gaacctgttttctgtatttaaggttttagaatgcaatttc

SEQ ID NO: 8 - DNA, 40 bp, Artificial
  synthetic primer
gaacttttcaaagttggcgtataacatagtatcgactttc

SEQ ID NO: 9 - DNA, 40 bp, Artificial
  synthetic primer
gaacctggatgaattgttttagtaccgagctcgctttttc

SEQ ID NO: 10 - DNA, 60 bp, Artificial
  synthetic primer
ttttgacttaactctagttcgtcatcgcatagatgatttagaaatcgccgtcgggcatgc

SEQ ID NO: 11 - DNA, 60 bp, Artificial
  synthetic primer
gcgccttgagcctggcgttcgtcatcgcatagatgatttagaaaggctacctgcccattc

SEQ ID NO: 12 - DNA, 60 bp, Artificial
  synthetic primer
gaccaccaagcgaaacgttcgtcatcgcatagatgatttagaaacaaccttaccagaggg

SEQ ID NO: 13 - DNA, 60 bp, Artificial
  synthetic primer
cgccccagctggcaatgttcgtcatcgcatagatgatttagaaaggccgcttgggtggag

SEQ ID NO: 14 - DNA, 60 bp, Artificial
  synthetic primer
aggctattcggctatggttcgtcatcgcatagatgatttagaaacggccggtagaaagga

SEQ ID NO: 15 - DNA, 40 bp, Artificial
  synthetic primer
gaacgccaggctcaaggcgcgcatgcccgacggcgatttc

SEQ ID NO: 16 - DNA, 40 bp, Artificial
  synthetic primer
gaacgtttcgcttggtggtcgaatgggcaggtagcctttc

SEQ ID NO: 17 - DNA, 40 bp, Artificial
  synthetic primer
gaacattgccagctggggcgccctctggtaaggttgtttc

SEQ ID NO: 18 - DNA, 40 bp, Artificial
  synthetic primer
gaaccatagccgaatagcctctccacccaagcggcctttc

SEQ ID NO: 19 - DNA, 30 bp, Artificial
  synthetic primer
tcctttctaccggccgtttctaaatcatct

SEQ ID NO: 20 - DNA, 40 bp, Artificial
  synthetic primer
tttggtctcatcctttctaccggccgtttctaaatcatct

SEQ ID NO: 21 - DNA, 60 bp, Artificial
  synthetic primer
aaaacaattcatccaggttcgtcatcgcatagatgatttagaaatcgccgtcgggcatgc

SEQ ID NO: 22 - DNA, 24 bp, Artificial
  synthetic primer
tagagttaagtcaaaacaaaaccc

SEQ ID NO: 23 - DNA, 20 bp, Artificial
  synthetic primer
gaaacggccggtagaaagga

SEQ ID NO: 24 - DNA, 46 bp, Artificial
  synthetic primer
tttggtctcagcgatgacgaactagagttaagtcaaaacaaaaccc

SEQ ID NO: 25 - DNA, 58 bp, Artificial
  synthetic primer
tttggtctcagttcgtcatcgcatagatgatttagaaacggccggtagaaaggagaag

SEQ ID NO: 26 - DNA, 24 bp, Artificial
  synthetic primer
tgagccgacattttattaccctct

SEQ ID NO: 27 - DNA, 21 bp, Artificial
  synthetic primer
ttacctgaaagccaatcgctg

SEQ ID NO: 28 - DNA, 46 bp, Artificial
  synthetic primer
tttggtctcatcgcatagatgatttagaaacggaattcaaggggac

SEQ ID NO: 29 - DNA, 60 bp, Artificial
  synthetic primer
aggtagcgcaggtgatgttcgtcatcgcatagatgatttagaaaatcgcgcgttacctcc

SEQ ID NO: 30 - DNA, 60 bp, Artificial
  synthetic primer
tgaacatcctctacaggttcgtcatcgcatagatgatttagaaagagaagtgaacttgtc

SEQ ID NO: 31 - DNA, 40 bp, Artificial
  synthetic primer
gaacatcacctgcgctacctgtccccttgaattccgtttc

SEQ ID NO: 32 - DNA, 40 bp, Artificial
  synthetic primer
gaacctgtagaggatgttcaggaggtaacgcgcgattttc

SEQ ID NO: 33 - DNA, 50 bp, Artificial
  synthetic primer
tttggtctcagaacttgaaattggtttatcgacaagttcacttctctttc

SEQ ID NO: 34 - DNA, 46 bp, Artificial
  synthetic primer
tttggtctcatcgcatagatgatttagaaatctccgcgcttgcttc

SEQ ID NO: 35 - DNA, 60 bp, Artificial
  synthetic primer
gcataatgcagattgagttcgtcatcgcatagatgatttagaaagtcactatgaccatgt

SEQ ID NO: 36 - DNA, 60 bp, Artificial
  synthetic primer
tgctttgtattgtgaagttcgtcatcgcatagatgatttagaaacccggattttgactgg

SEQ ID NO: 37 - DNA, 60 bp, Artificial
  synthetic primer
cgaaatgtagaagatagttcgtcatcgcatagatgatttagaaacggaattcaaggggac

SEQ ID NO: 38 - DNA, 40 bp, Artificial
  synthetic primer
gaactcaatctgcattatgcgaagcaagcgcggagatttc

SEQ ID NO: 39 - DNA, 40 bp, Artificial
  synthetic primer
gaacttcacaatacaaagcaacatggtcatagtgactttc

SEQ ID NO: 40 - DNA, 40 bp, Artificial
  synthetic primer
gaactatcttctacatttcgccagtcaaaatccgggtttc

SEQ ID NO: 41 - DNA, 27 bp, Artificial
  synthetic primer
ggagttctgaggtcattactggatcta

SEQ ID NO: 42 - DNA, 20 bp, Artificial
  synthetic primer
caaatgtacggccagcaacg

SEQ ID NO: 43 - DNA, 20 bp, Artificial
  synthetic primer
agcagctgagagcctgaatg

SEQ ID NO: 44 - DNA, 21 bp, Artificial
  synthetic primer
acatgccagcacttaatctga

SEQ ID NO: 45 - DNA, 45 bp, Artificial
  synthetic primer
tcagattaagtgctggcatgtgcacccaatccctaacattaaaca

SEQ ID NO: 46 - DNA, 20 bp, Artificial
  synthetic primer
ggttcgggcacctcatcatt

SEQ ID NO: 47 - DNA, 20 bp, Artificial
  synthetic primer
acagggcagccattaactga

SEQ ID NO: 48 - DNA, 21 bp, Artificial
  synthetic primer
tctgagactgtagcctacgca

SEQ ID NO: 49 - DNA, 44 bp, Artificial
  synthetic primer
tgcgtaggctacagtctcagaacgaagttatgtgccacaagaaa

SEQ ID NO: 50 - DNA, 20 bp, Artificial
  synthetic primer
tcagacgcaagcgtgaagat

SEQ ID NO: 51 - DNA, 20 bp, Artificial
  synthetic primer
gcctcctaaaattgggggct

SEQ ID NO: 52 - DNA, 20 bp, Artificial
  synthetic primer
cttggttctgcattgggtgc

SEQ ID NO: 53 - DNA, 20 bp, Artificial
  synthetic primer
gacttgcgtaggcttggact

SEQ ID NO: 54 - DNA, 20 bp, Artificial
  synthetic primer
gcatgtcatggtttggtggg

SEQ ID NO: 55 - DNA, 20 bp, Artificial
  synthetic primer
atgaacgcgatcattgcagc

SEQ ID NO: 56 - DNA, 20 bp, Artificial
  synthetic primer
tacggccaattgatcaccca

SEQ ID NO: 57 - DNA, 59 bp, Artificial
  synthetic primer
tttggtctcataagaactttaaataatttctactgttgtagatcggcgtcaatacggga

SEQ ID NO: 58 - DNA, 68 bp, Artificial
  synthetic primer
taataccgcgccacatgtctaagaactttaaataatttctactgttgtagatggagctgaatgaagcc

SEQ ID NO: 59 - DNA, 68 bp, Artificial
  synthetic primer
ataccaaacgacgagcgtctaagaactttaaataatttctactgttgtagatctcccgtatcgtagtt

SEQ ID NO: 60 - DNA, 68 bp, Artificial
  synthetic primer
atctacacgacggggagtctaagaactttaaataatttctactgttgtagatagccggaagggccgag

SEQ ID NO: 61 - DNA, 64 bp, Artificial
  synthetic primer
tttggtctcagtctaagaactttaaataatttctactgttgtagatcggccggtagaaaggaca

SEQ ID NO: 62 - DNA, 41 bp, Artificial
  synthetic primer
tttggtctcacttagactagagttaagtcaaaacaaaaccc

SEQ ID NO: 63 - DNA, 40 bp, Artificial
  synthetic primer
agacatgtggcgcggtattatcccgtattgacgccgatct

SEQ ID NO: 64 - DNA, 40 bp, Artificial
  synthetic primer
agacgctcgtcgtttggtatggcttcattcagctccatct

SEQ ID NO: 65 - DNA, 40 bp, Artificial
  synthetic primer
agactccccgtcgtgtagataactacgatacgggagatct

SEQ ID NO: 66 - DNA, 50 bp, Artificial
  synthetic primer
tttggtctcaagaccaggaccacttctgcgctcggcccttccggctatct

SEQ ID NO: 67 - DNA, 32 bp, Artificial
  CRISPR spacers
ggtcgatcagggaggatatcggggaagaacag

SEQ ID NO: 68 - DNA, 32 bp, Artificial
  CRISPR spacers
gtcgatactatgttatacgccaactttgaaaa

SEQ ID NO: 69 - DNA, 32 bp, Artificial
  CRISPR spacers
ttgcattctaaaaccttaaatacagaaaacag

SEQ ID NO: 70 - DNA, 32 bp, Artificial
  CRISPR spacers
aagcgagctcggtactaaaacaattcatccag

SEQ ID NO: 71 - DNA, 32 bp, Artificial
  CRISPR spacers
tcgccgtcgggcatgcgcgccttgagcctggc

SEQ ID NO: 72 - DNA, 32 bp, Artificial
  CRISPR spacers
caaccttaccagagggcgccccagctggcaat

SEQ ID NO: 73 - DNA, 32 bp, Artificial
  CRISPR spacers
ggctacctgcccattcgaccaccaagcgaaac

SEQ ID NO: 74 - DNA, 32 bp, Artificial
  CRISPR spacers
ggccgcttgggtggagaggctattcggctatg

SEQ ID NO: 75 - DNA, 32 bp, Artificial
  CRISPR spacers
tctccgcgcttgcttcgcataatgcagattga

SEQ ID NO: 76 - DNA, 32 bp, Artificial
  CRISPR spacers
gtcactatgaccatgttgctttgtattgtgaa

SEQ ID NO: 77 - DNA, 32 bp, Artificial
  CRISPR spacers
cccggattttgactggcgaaatgtagaagata

SEQ ID NO: 78 - DNA, 32 bp, Artificial
  CRISPR spacers
cggaattcaaggggacaggtagcgcaggtgat

SEQ ID NO: 79 - DNA, 32 bp, Artificial
  CRISPR spacers
atcgcgcgttacctcctgaacatcctctacag

SEQ ID NO: 80 - DNA, 32 bp, Artificial
  CRISPR spacers
gagaagtgaacttgtcgataaaccaatttcaa

SEQ ID NO: 81 - DNA, 32 bp, Artificial
  CRISPR spacers
taggggaaagcctactagccggagtgttgcga

SEQ ID NO: 82 - protein, 263 aa, Artificial
  CRISPR vector
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 83 - DNA, 3872 bp, Artificial
  CRISPR vector
tgagccgacattttattaccctcttatcaaaccgtacctttcacataacgaatgaatgaataccgtacatggagtgcggccaacccacagcgaacatcatatttcgcatccatcaccgtacggttttccgttttaagctctgcccatgatctatcatggaaataacggctaatgatcacctgcatccactcaagtgtcgtttcactgtctgtaccattaataatatccagtactaaacgttgtacggcacgagcttcattatcgttaatctgacacgacactttgtgacgtatagcttgttgtactccttgagcatcacaaaggtaataagcaataagtttagctcgatctttcttctttacacgtaccttggctttcttcattgcaatagcaatcgggctatcggaatactgtccaccacgacaagaacgttgccacgctccacattggcgtaaccaatctggcaaatcatacttgctccaatccaccgtctgcataatgtgcactgctgtattcatctcatcacctaatttgtttcaagttaaattttataagcgttattgttttatggttctgcctgctcctctaccgatctaaaacgacaagtttcgagataatccagtactcgaactgcaccgcgtttaccgtgtcggtttttcactacaatcagctctgtgattcccatcggtttggttgagtcttctggatcggttagggggttaacaaggatgatctggtctgcatcttgctcgatttgtccagattctttgatatctgatgctttaggacgtttgcccttctctgcctcacggttgagctgtaccagtgcaatgacaggacattcaaactctttcgccatggattttaattcacggctgatggaactgacttcctgaaagcgatctttcttgctcgggtctctgagcagttgtaaataatccacgatgatgcagcccaattccttgtaacggcgtttggctcgacgtgcataggaacggacctcactcaagtgattcataacgaagtatttttactcattaaaagcttatataattgatatcaagggttttgttttgacttaactctagttcgtcatcgcatagatgatttagaaaggtcgatcagggaggatatcggggaagaacaggttcgtcatcgcatagatgatttagaaattgcattctaaaaccttaaatacagaaaacaggttcgtcatcgcatagatgatttagaaagtcgatactatgttatacgccaactttgaaaagttcgtcatcgcatagatgatttagaaaaagcgagctcggtactaaaacaattcatccaggttcgtcatcgcatagatgatttagaaatcgccgtcgggcatgcgcgccttgagcctggcgttcgtcatcgcatagatgatttagaaaggctacctgcccattcgaccaccaagcgaaacgttcgtcatcgcatagatgatttagaaacaaccttaccagagggcgccccagctggcaatgttcgtcatcgcatagatgatttagaaaggccgcttgggtggagaggctattcggctatggttcgtcatcgcatagatgatttagaaacggccggtagaaaggagaagcttactagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcggttcagggcagggtcgttaaatagccgcttatgtctattgctggtttaccggtttattgactaccggaagcagtgtgaccgtgtgcttctcaaatgcctgaggtttcagcaaaaaacccctcaagacccgtttagaggccccaaggggttatgctagttattgctcagcggtggcagcagcctaggttaattaagctgcgctagtagacgagtccatgtgctggcgttcaaatttcgcagcagcggtttctttaccagactcgacaagcttactagagtgttcatattgacctcgcttagtgtggttaatacgccgcttcttgttactgcaagaggcggtttttttatgggtgtacacatgactgcacctgttgattcagttcagatagtgctttgtgcatctcatggatgactctggccatatccagtgcttcgccttgggtaattcgcccatcggccatcatttctttaaacagtgctgatatatcgcccttcttgatgcctatgcataggaaggtatccatcagactggtatctcgctggctctcgggtatgtctggcaggtcaattgccacctttccgagtcgtgcacacatttcctgcaatatccgatagtcccctgtaatctccatcagcttgactgcctcgagcaatgtaatgtgatgggtatgtgtgtttgggttgaccttgctattgagcaccgcagggcttttgatgcctaaacgtgatgcaagtgcagatgcaccacccagaaagtcgtgaacggtgtgataggcagcatctaatatgttcatggcgggttcctttgaacgtgtttattagatgggtgctgacataagattggatttatggttaggacgtaattcaatccaaatatcttcgtaatcatctggaaataaatctttgcgagtgcatagacctttatcttcagcaattacagctaagcggattttgcgatctctgggaattgctttccatccacttacggatgcagcggtgatacctaaaaatctagcaacagcagttacaccacctaaaagctcaataaattgatcatcagtcatgttgatctcctaattttattgcctcaattattaggtattccttatattttatcaataggaataccttatttattttatgttaggatttcctaatagactaggtaagatcatgaaaacattagctgaacgacttaaatatgcgatggaaattttgccacctaagaaaatcaagggtgtcgaacttgctcgtgtagttggagttaaaccaccatctgtcagcgattggctttcaggtaa

SEQ ID NO: 84 - protein, 232 aa, Artificial
  CRISPR vector
MAIKNTKARNFGFLLYPDSIPNDWKEKLESLGVSMAVSPLHDMDEKKDKDTWNSSDVIRNGKHYKKPHYHVIYIARNPVTIESVRNKIKRKLGNSSVAHVEILDYIKGSYEYLTHESKDAIAKNKHIYDKKDILNINDFDIDRYITLDESQKRELKNLLLDIVDDYNLVNTKDLMAFIRLRGAEFGILNTNDVKDIVSTNSSAFRLWFEGNYQCGYRASYAKVLDAETGEIK

SEQ ID NO: 85 - protein, 53 aa, Artificial
  CRISPR vector
MVISESKKRVMISLTKEQDKKLTDMAKQKGFSKSAVAALAIEEYARKESEQKK

SEQ ID NO: 86 - protein, 69 aa, Artificial
  CRISPR vector
MGGKEANFASVLRPPIKCRVPIFVPKTLYPNWLKGLRGFSIANESPTFSPTFFINLYLSSFIVVFMITK

SEQ ID NO: 87 - protein, 263 aa, Artificial; identical to SEQ ID NO: 82
  CRISPR vector

SEQ ID NO: 88 - DNA, 3162 bp, Artificial
  CRISPR vector
tagaaaggagaagcttactagtagcggccgctgcaggcctcagggcccgatcgatgccgccgcttaattaattaatccagaggcatcaaataaaacgaaaggctcagtcgaaagactgggcctttcgttttatctgttgtttgtcggtgaacgctctcctgagtaggacaaatccgccgccctagacctagtgtcattttatttcccccgtttcagcatcaagaacctttgcataacttgctctatatccacactgataattgccctcaaaccataatctaaaggcgctagagtttgttgaaacaatatcttttacatcattcgtatttaaaattccaaactccgctcccctaaggcgaataaaagccattaaatcttttgtatttaccaaattatagtcatccactatatctaagagtaaattcttcaattctcttttttggctttcatcaagtgttatatagcggtcaatatcaaaatcattaatgttcaaaatatcttttttgtcgtatatatgtttattcttagcaatagcgtcctttgattcatgagtcaaatattcatatgaacctttgatataatcaagtatctcaacatgagcaactgaactattccccaattttcgcttaatcttgttcctaacgctttctattgttacaggatttcgtgcaatatatataacgtgatagtgtggttttttatagtgctttccatttcgtataacatcactactattccatgtatctttatcttttttttcgtccatatcgtgtaaaggactgacagccatagatacgcccaaactctctaatttttccttccaatcattaggaattgagtcaggatataataaaaatccaaaatttctagctttagtatttttaatagccatgatataattaccttatcaaaaacaagtagcgaaaactcgtatccttctaaaaacgcgagctttcgcttattttttttgttctgattcctttcttgcatattcttctatagctaacgccgcaaccgcagattttgaaaaacctttttgtttcgccatatctgttaattttttatcttgctcttttgtcagagaaatcataactctttttttcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggcactcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgcctagtgcttggattctcaccaataaaaaacgcccggcggcaaccgagcgttctgaacaaatccagatggagttctgaggtcattactggatctacaagtgattcataacgaagtatttttactcattaaaagcttatataattgatatcaagggttttgttttgacttaactctagttcgtcatcgcatagatgatttagaaatctccgcgcttgcttcgcataatgcagattgagttcgtcatcgcatagatgatttagaaagtcactatgaccatgttgctttgtattgtgaagttcgtcatcgcatagatgatttagaaacccggattttgactggcgaaatgtagaagatagttcgtcatcgcatagatgatttagaaacggccggtagaaaggagaagcttactagctatttgtttatttttctaaatacattcaaatatgtatccgctcatgagacaataaccctgataaatgcttcaataatattgaaaaaggaagagtatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgaggggccgcaagatccggccacgatgacccggtcgtcggttcagggcagggtcgttaaatagccgcttatgtctattgctggtttaccggtttattgactaccggaagcagtgtgaccgtgtgcttctcaaatgcc

SEQ ID NO: 89 - DNA, 62 bp, Artificial
  Synthetic primer
tttggtctcagtctaagaactttaaataatttctactgttgtagatcggcgtcaatacggga

SEQ ID NO: 90 - DNA, 68 bp, Artificial; identical to SEQ ID NO: 58
  Synthetic primer

SEQ ID NO: 91 - DNA, 68 bp, Artificial
  Synthetic primer
ataccaaacgacgagcgtctaagaactttaaataatttctactgttgtagatagccggaagggccgag

SEQ ID NO: 92 - DNA, 36 bp, Artificial
  Synthetic primer
atgtggcgcggtattatcccgtattgacgccgatct

SEQ ID NO: 93 - DNA, 36 bp, Artificial
  Synthetic primer
gctcgtcgtttggtatggcttcattcagctccatct

SEQ ID NO: 94 - DNA, 46 bp, Artificial
  Synthetic primer
tttggtctcacaggaccacttctgcgctcggcccttccggctatct

SEQ ID NO: 95 - DNA, 68 bp, Artificial
  Synthetic primer
tttggtctcacctggtctaagaactttaaataatttctactgttgtagatcggccggtagaaaggaca

SEQ ID NO: 96 - DNA, 38 bp, Artificial
  Synthetic primer
tttggtctcaagactagagttaagtcaaaacaaaaccc

(19) United States
(12) Patent Application Publication
(10) Pub. No.: US 2024/0141399 A1
(43) Pub. Date: May 2, 2024

(54) METHODS FOR GENERATING A CRISPR ARRAY

(71) Applicant: THE REGENTS OF THE UNIVERSITY OF CALIFORNIA, Oakland, CA (US)

(72) Inventors: Robert Matthew Cooper, San Diego, CA (US); Jeff Hasty, Encinitas, CA (US)

(21) Appl. No.: 18/278,695

(22) PCT Filed: Feb. 28, 2022
(86) PCT No.: PCT/US2022/018107

§ 371 Date: Aug. 24, 2023

Related U.S. Application Data
(60) Provisional application No. 63/155,103, filed on Mar. 1, 2021.

(51) Int. Cl. C12P 19/34

(52) U.S. Cl. CPC C12P 19/34

(57) ABSTRACT

Provided herein are methods for generating multiplex CRISPR arrays based on annealing and ligating single-stranded DNA oligonucleotides using bridge oligonucleotides. The methods described herein include providing a first oligonucleotide comprising a CRISPR repeat sequence or a portion thereof, and a first portion of a first spacer sequence at its 3′ end; providing a second oligonucleotide comprising, from 5′ to 3′, a second portion of the first spacer sequence, the CRISPR repeat sequence, and a first portion of a second spacer sequence; providing a bridge oligonucleotide comprising a sequence substantially complementary to the first spacer sequence; allowing the first oligonucleotide and the second oligonucleotide to hybridize with the bridge oligonucleotide; and ligating the first and second oligonucleotide.

### CROSS-REFERENCE TO RELATED APPLICATIONS

This application claims priority to U.S. Patent Application Ser. No. 63/155,103, filed Mar. 1, 2021, which is incorporated herein by reference in its entirety.

### STATEMENT OF GOVERNMENT SUPPORT

This invention was made with government support under GM085764 awarded by the National Institutes of Health. The government has certain rights in the invention.

### BACKGROUND

One of the key advantages of CRISPR-Cas systems for biotechnology is that their nucleases can use multiple guide RNAs in the same cell. However, multiplexing with CRISPR-Cas9 and its homologs presents various technical challenges, such as very long synthetic targeting arrays and time-consuming assembly. Recently, other CRISPR associated, single-effector nucleases such as Cas12a have been shown to process their own CRISPR arrays, enabling the use of much more compact natural arrays. However, these highly repetitious arrays can be difficult to synthesize commercially or assemble in the lab. Therefore, improved compositions and methods for assembling multiple CRISPR arrays are needed.

### SUMMARY

Provide herein are methods of generating a CRISPR array, the method comprising: providing a first oligonucleotide comprising a CRISPR repeat sequence or a portion thereof, and a first portion of a first spacer sequence at its 3′ end; providing a second oligonucleotide comprising, from 5′ to 3′, a second portion of the first spacer sequence, the CRISPR repeat sequence, and a first portion of a second spacer sequence; providing a bridge oligonucleotide comprising a sequence substantially complementary to the first spacer sequence; allowing the first oligonucleotide and the second oligonucleotide to hybridize with the bridge oligonucleotide; and ligating the first and second oligonucleotide. In some embodiments, the first oligonucleotide further comprises, at its 5′ end, a flanking sequence. In some embodiments, the first oligonucleotide comprises, from 5′ to 3′, a flanking sequence, a CRISPR repeat sequence or a portion thereof, and a first portion of a first spacer sequence. In some embodiments, the flanking sequence comprises a portion of a sequence of a vector. In some embodiments, the first oligonucleotide further comprises, at its 5′ end, a portion of a third spacer sequence. In some embodiments, the first oligonucleotide comprises, from 5′ to 3′, a portion of a third spacer sequence, a CRISPR repeat sequence or a portion thereof, and a first portion of a first spacer sequence. In some embodiments, the bridge oligonucleotide further comprises a sequence substantially complementary to a portion of the CRISPR repeat sequence at its 5′ or 3′ end. In some embodiments, the portion of the CRISPR repeat sequence comprises about 1 to about 10 nucleotides. In some embodiments, the bridge oligonucleotide comprises, from 5′ to 3′, a sequence substantially to a first portion of the CRISPR repeat sequence, the sequence substantially complementary to the first spacer sequence, and a sequence substantially complementary to a second portion of the CRISPR repeat sequence. In some embodiments, the first and/or second portion of the CRISPR repeat sequence comprises about 1 to about 10 nucleotides. In some embodiments, each of the first and second oligonucleotides comprises about 40 to about 70 nucleotides. In some embodiments, each of the first and second oligonucleotides comprises about 55 to about 65 nucleotides. In some embodiments, the CRISPR repeat sequence comprises about 15 to about 36 nucleotides. In some embodiments, the bridge oligonucleotide comprises about 30 to about 50 nucleotides. In some embodiments, each of the first portion of the first spacer sequence, the second portion of the first spacer sequence, and the first portion of the second spacer sequence comprises about 5 to about 20 nucleotides. In some embodiments, the first spacer sequence comprises a first target site in a target gene, and the second spacer sequence comprises a second target site in the target gene. In some embodiments, the first spacer sequence comprises a target site in a first target gene, and the second spacer sequence comprises a target site in a second target gene. In some embodiments, the bridge oligonucleotide is used at a ratio of between about 2:1 and about 3:1 by molarity in relation to a mixture of the first and second oligonucleotides. In some embodiments, the amount of the first and second oligonucleotides in the mixture are about equal. In some embodiments, the first oligonucleotide, the second oligonucleotide, and the bridge oligonucleotide are DNA oligonucleotides. In some embodiments, ligating the first and second oligonucleotides comprises using a DNA ligase. In some embodiments, ligating the first and second oligonucleotides is carried out at about 25° C. to about 45° C. In some embodiments, ligating the first and second oligonucleotides is carried out at about 37° C. In some embodiments, the methods comprise ligating three or more oligonucleotides. In some embodiments, the method further comprises generating a strand complementary to the ligated first and second oligonucleotide, wherein the complementary strand comprises the bride oligonucleotide, thereby generating a double-strand construct. In some embodiments, the method further comprising PCR amplification of the double-strand construct. In some embodiments, the method further comprising inserting the PCR amplified construct into a vector.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIGS. 1A-1D show synthetic A. baylyi CRISPR arrays blocking gene acquisition via natural competence. FIG. 1A shows the endogenous, Type I-F CRISPR locus in A. baylyi. FIGS. 1B-1D show cells containing individual spacer arrays (T1, T2, B1, or B2), a 4-spacer multiplex array including all individual spacers, or a random spacer were naturally transformed with the self-replicating plasmid pBAV-K1 (FIG. 1B), the integrating linear DNA Vgr4-K1 (FIG. 1C), or the non-targeted, integrating linear DNA Vgr4-K2 (FIG. 1D). The fraction of cells acquiring kanamycin resistance is shown on a log scale. Data includes 2 experimental replicates, each with 3 measurement replicates, error bars indicate propagated standard deviations (see Methods), and limits of detection were roughly 10⁻⁶. Statistical comparison to the random spacer was performed using multiple comparison analysis (Methods): *=p<0.01, **=p<10⁻⁶.

FIGS. 2A-2E show a strategy for assembling multiplex, natural CRISPR arrays. Assembly strategy for a sample 3-spacer CRISPR array to be inserted into a vector using Gibson assembly or fusion PCR (FIG. 2A), or Golden Gate assembly (FIG. 2B). Each strategy shows the desired end product, the top and bottom oligos used for array annealing and ligation, and the PCR amplicons for insertion into a vector. Single-stranded primers (oligos) are shown as arrows pointing 5′ to 3′. Primers used for Golden Gate assembly (denoted “GG”) have an additional Golden Gate tail appended to their 5′ ends. FIG. 2C shows PCR amplified 9-spacer arrays using the Gibson (left) and Golden Gate (right) strategies. Colony PCR screening of E. coli clones for 9-spacer arrays inserted using Golden Gate (FIG. 2D) and Gibson (FIG. 2E) strategies, where the correct length is 914 bp. The ladder on all gels has 100 bp increments, with the 1 kb band marked by an asterisk.

FIGS. 3A-3E show multiplex array assembly optimization. Protocol optimizations were performed using a 6×IS-CRA array and inserted into pBAV using Golden Gate assembly. FIG. 3A shows including the Repeat_RC oligo increases incorrect, higher-molecular-weight smearing (left 3 vs right 3 lanes), and 100 μM stock oligos (lanes 1 and 5) work better than 33 μM (lanes 2 and 6) or 10 μM (lanes 3 and 7) stock solutions. The center (lane 4) is a 100 bp ladder. FIG. 3B shows annealing and ligation is most efficient using 3 parts bottom oligos to 1 part top oligos. The lanes from left to right are ligations using 1:1, 3:1, and 10:1 ratios of bottom oligos to top oligos, followed by a 100 bp ladder. FIG. 3C shows PCT amplification of the resulting ligation improves yield of the correct-sized product. FIG. 3D shows Golden Gate assembly directly from ligation products yielded no correct-sized arrays out of 36 tested clones. All of 6 sequenced clones were correct at the 3′ end, but truncated at the 5′ end of the array. FIG. 3E, as for FIG. 3D, but the ligation product was PCT amplified and gel extracted before inserting into the vector. 16 of 25 colonies were the correct size, and all incorrect clones had Ox arrays (a single repeat only).

FIGS. 4A and 4B show detailed multiplex natural CRISPR array assembly. A more detailed version of FIGS. 2A-2B showing DNA sequences for the 3×BAP CRISPR array. The two array assembly strategies are for insertion into a vector using Gibson assembly or fusion PCR (FIG. 4A) or Golden Gate assembly (FIG. 4B). Primers used for Golden Gate assembly (denoted “GG” in FIG. 4B) have an additional BsaI site-containing tail appended to their 5′ ends that is not shown, specifically, TTTGGTCTCA.

FIGS. 5A-5D are diagrams showing the effectiveness of 4-spacer and 8-spacer natural arrays inserted into the A. baylyi genome against the genomically integrating DNA. Cells containing no exogenous CRISPR arrays (WT), 4-spacer arrays targeting kan1 and kan2, and an 8-spacer array targeting both kan genes (x-axis tick labels) were incubated with linear, genomically integrating DNA. Donor DNA constructs included Vgr4-Kan1 (FIG. 5A), Vgr4-Kan2 (FIG. 5B), both kan constructs (FIG. 5C), or a non-targeted beta-lactamase gene (FIG. 5D). Data includes 2 experimental replicates, each with 3 measurement replicates, error bars indicate propagated standard deviations, and limits of detection were roughly 10⁻⁶. **=p<10⁻⁷.

FIGS. 6A-6F are gel images showing the deletion of bap and CRAΦ in A. baylyi using multi-spacer arrays. Arrows indicate the expected bands for correct genomic edits, and asterisks indicate the 1 kb band of the ladder (not counted in lane numbering). FIG. 6A shows PCR screening of 3×BAP (lanes 1-8) and 6×CRA-BAP (lanes 9-16) arrays in pBAV, cloned into E. coli. FIG. 6B shows PCR screening of 2 markerless bap deletions in A. baylyi using pBAV-CRISPR_{3×BAP}. FIGS. 6C-6F show PCR screening of markerless bap and double CRAΦ, bap deletions in A. baylyi using non-clonal, linear PCR products from array assembly. FIG. 6C shows multiplex 3×BAP (lanes 1-8) and 6×CRA-BAP (lanes 9-16) arrays. FIG. 6D shows bap deletion screening for the same clones as in FIG. 6C. The deletion and wild type amplicons are roughly 4.5 and 12 kb, respectively. FIG. 6E shows CRAΦ deletion screening for the clones in lanes 9-16 of FIGS. 6C and 6D. Product was only expected for CRAΦ deletion. FIG. 6F, as in 6E, but circular CRAΦ phage screening. The 3 kb product was only expected if CRAΦ was present in its excised, circular episome form.

FIGS. 7A and 7B show assembly of a 4-spacer Cas12a array. FIG. 7A shows the design and oligonucleotides for a 4-spacer FnCas12a CRISPR array, to be inserted into the vector using Golden Gate assembly. This is analogous to FIG. 2B for A. baylyi arrays. All oligos denoted by GG also contain a 5′ Golden Gate tail. FIG. 7B shows screening of 8 clones for the 4-spacer array, of which all had the desired 603 bp product. The primer pair hybridized to the backbone of the vector, outside the inserted CRISPR array. The ladder on the end lanes contains 100 bp increments up to 1 kb.

FIG. 8 shows array assembly strategy for insertion into the vector using a Golden Gate approach.

FIG. 9 shows array assembly strategy including sequence.

FIG. 10 shows sample PCR screen for 16 clones of a 9-spacer CRISPR array. The ladder on the end lanes goes from 100 bp to 1 kb in increments of 100 bp. The expected length is about 900 bp, with 11 of 16 clones having the correct number of spacers.

### DETAILED DESCRIPTION

The present disclosure provides methods of generating multiplex CRISPR arrays based on annealing and ligating single-stranded DNA oligonucleotides using bridge oligonucleotides. The methods described herein include providing a first oligonucleotide comprising a CRISPR repeat sequence or a portion thereof, and a first portion of a first spacer sequence at its 3′ end; providing a second oligonucleotide comprising, from 5′ to 3′, a second portion of the first spacer sequence, the CRISPR repeat sequence, and a first portion of a second spacer sequence; providing a bridge oligonucleotide comprising a sequence substantially complementary to the first spacer sequence; allowing the first oligonucleotide and the second oligonucleotide to hybridize with the bridge oligonucleotide; and ligating the first and second oligonucleotide.

CRISPR (clustered regularly interspaced short palindromic repeats)-Cas systems are adaptive immunity mechanisms that protect bacteria and archaea against invading nucleic acids, generally by detecting and cutting or degrading defined target sequencesl. CRISPR-Cas systems include Cas (CRISPR-associated) proteins, as well as their eponymous arrays of short direct repeats that alternate with similarly short DNA spacers. The spacer array is transcribed into a long pre-crRNA, which is then processed into individual crRNAs (CRISPR RNAs), each composed of a single spacer that is complementary to a particular nucleic acid target, and often a hairpin handle derived from a repeat. These crRNAs bind Cas effector proteins, such as Cas9, or multi-protein complexes, such as CASCADE. Once bound, they guide the effector to complementary DNA or RNA, depending on the system, which the effectors often cleave and/or degrade.

Spacer multiplexing is beneficial for many of the applications of CRISPR-mediated DNA cleavage, including, e.g. precise genome engineering, genetic circuits, targeted bacterial strain removal. Spacer multiplexing is also beneficial for self-spreading CRISPR constructs. Self-spreading CRISPR constructs have been used to quickly generate homozygous diploid knock-outs (the mutagenic chain reaction), and preliminary work suggests they could re-engineer entire populations through biased inheritance; i.e., gene drives or active genetics.

Targeting multiple sites on the same gene improves both mutagenesis and gene regulation, cleaving multiple target sites prevents emergence of resistant alleles, and multiple genes can be edited simultaneously.

While natural CRISPR arrays are inherently multiplex—some including hundreds of spacers—multiplexing in synthetic biology applications has been comparatively limited. One reason is that constructing synthetic multiplex CRISPR arrays is technically challenging due to their extensive repetition. Addressing this difficulty, several strategies have been developed to assemble tandem arrays of synthetic sgRNA (single guide RNA) transcriptional units, but these are limited in array size or required time-consuming, sequential cloning for each additional spacer. Recently, single-promoter sgRNA arrays have been shown to be assembled using tRNAs to direct processing and release of individual sgRNAs.

The majority of early work has used the single effector nuclease Cas9. Cas9 itself is very simple to port to other organisms, because it requires only a single gene. However, the simplicity of the coding gene comes at the expense of greater sequence length and complexity for the targeting array. Cas9 does not process its own arrays and requires a trans-activating CRISPR RNA (tracrRNA), so to port it to other organisms, scientists usually use synthetic tracrRNA-guide RNA (gRNA) fusions called single guide RNAs (sgRNAs), which are each expressed from an independent transcriptional unit. The resulting array complexity rapidly becomes a problem when using more than one guide RNA. Performing multiplex targeting with Cas9 often requires many cloning steps and/or long sgRNA arrays that can exceed the length capacity of viral vectors.

However, the more recent discovery that other single-protein CRISPR effectors, including Cas12a (Cpf1) and Cas13a (C2c2), can process natural arrays without tracrRNA means that natural, multiplex CRISPR arrays can be used in non-native hosts as easily as sgRNAs. In comparison to artificial sgRNA arrays, natural CRISPR arrays have several advantages for multiplexing. Natural arrays are much more compact, making them easier to package and deliver. Natural arrays also have a particular advantage for applications in prokaryotes, many of which already have their own endogenous CRISPR-Cas systems that can be retargeted using synthetic spacers. Such a system can be used to limit horizontal gene transfer, a major contributor to multi-drug resistance and pathogenicity.

The CRISPR-Cas12 system, for example, was shown to process its own CRISPR array using the same single enzyme cleaves its target. This system allows the best of both worlds for synthetic multiplexing applications—a compact single gene paired with a compact natural CRISPR array. Unfortunately, the eponymous palindromic repetition of natural CRISPR arrays makes longer multiplex arrays difficult for commercial providers to synthesize and for individual researchers to assemble. Thus, while Cas12 solves the array length problem of synthetic Cas9 systems, multiplexing with longer natural CRISPR arrays has still required either time-consuming cloning with each spacer added to the array one at a time, or sequence modifications to the ends of the spacers.

The signature palindromic repeats significantly complicate assembly of natural CRISPR arrays. This problem is particularly important because spacer design rules are not completely accurate even for the best studied Cas nucleases, so developing good arrays can require building and testing multiple designs. Recent approaches for assembling multiplex natural arrays have been limited to just a few spacers, imposed sequence constraints, or required sequential, time-consuming cloning steps for each additional spacer. Multiplex arrays can be assembled using very long single-stranded oligos (e.g., 180 nt), but these become significantly more expensive and unreliable as their length surpasses 60 nt. Another option is double-stranded DNA synthesis, but this can also be unreliable or require slower, more expensive cloned gene services. Such double-stranded DNA synthesis often takes longer or fails for sequences containing repetition and/or secondary structure, both of which are defining features of CRISPR arrays. Primed adaptation can generate multiplex arrays using the endogenous adaptation mechanism, but the results are stochastic, not designed. A recent one-pot method enables rapid assembly of nearly-natural CRISPR arrays, but this still requires trimming the 3′ ends of spacers. This makes the method incompatible with systems that do not trim their spacers and thus require sequence complementarity throughout, including the most prevalent Type I systemsl. Array assembly therefore remains a key challenge in the field.

A “target gene” as used herein can include nucleotide sequence that can include a “target site”. The “spacer sequence” within an oligonucleotide can include a nucleotide sequence within a target gene. The spacer sequence can be designed, for instance, to comprise the sequence of any target site or a portion thereof.

“Binding” as used herein can refer to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it means that the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. Kd is dependent on environmental conditions, e.g., pH and temperature, as is known by those in the art.

The terms “hybridizing” or “hybridize” can refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences or segments of sequences are “substantially complementary” if at least 80% of their individual bases are complementary to one another.

### I. Oligonucleotides

The present disclosure provides methods of generating CRISPR arrays, using bridge oligonucleotide mediated ligation of two or more oligonucleotides. A bridge oligonucleotide can anneal with a first and a second oligonucleotide and mediates ligation of the first and second oligonucleotides at a ligation site between the first and second oligonucleotide.

The first oligonucleotide can include a CRISPR repeat sequence or a portion thereof, and a first portion of a first spacer sequence at its 3′ end. The first oligonucleotide can further include, at its 5′ end a flanking sequence or a portion of a third spacer sequence. For example, the first oligonucleotide can include, from 5′ to 3′, a flanking sequence, a CRISPR repeat sequence or a portion thereof, and a first portion of a first spacer sequence. The flanking sequence can include a portion of the sequence of a vector. Any suitable vectors known in the art are contemplated herein, for example, the pBAV1k vector (Addgene #26702). The flanking sequence can also include an adaptor sequence suitable for Golden Gate cloning. The adaptor sequence can include a restriction enzyme (e.g. any Golden Gate compatible restriction enzyme known in the art) target site. In another example, the first oligonucleotide can include, from 5′ to 3, a portion of a third spacer sequence, a CRISPR repeat sequence or a portion thereof, and a first portion of a first spacer sequence.

The second oligonucleotide can include, from 5′ to 3′, a second portion of the first spacer sequence, a CRISPR repeat sequence, and a first portion of a second spacer sequence.

The bridge oligonucleotide can include a sequence substantially complementary to the first spacer sequence. The bridge oligonucleotide can hybridize with the first and second oligonucleotides to form a complex. In the complex, the first and second oligonucleotides are positioned favorably for ligation at a ligation site present between the first and second oligonucleotides. In some instances, the bridge oligonucleotide further includes a sequence substantially complementary to a portion of a CRISPR repeat sequence at its 5′ or 3′ end. The portion of the CRISPR repeat sequence comprises about 1 to about 10 nucleotides (e.g. 2, 3, 4, 5, 6, 7, 8, or 9 nucleotides). For example, the bridge oligonucleotide can include from 5′ to 3′, a sequence substantially complementary to a first portion of a CRISPR repeat sequence, the sequence substantially complementary to the first spacer sequence, and a sequence substantially complementary to a second portion of a CRISPR repeat sequence. The first and/or second portion of the CRISPR repeat sequence can include about 1 to about 10 nucleotides (e.g. 2, 3, 4, 5, 6, 7, 8, or 9 nucleotides). In some embodiments, the first oligonucleotide, the second oligonucleotide, and the bridge oligonucleotide are DNA oligonucleotides.

A CRISPR repeat sequence refers to a repetitive sequence found within a CRISPR locus (naturally-occurring in a bacterial genome or plasmid) that are interspersed with the spacer sequences. A CRISPR repeat sequence disclosed herein can bind to a Cas protein (e.g. any of the Cas proteins disclosed herein or known in the art). It is well known that one would be able to infer the CRISPR repeat sequence of a corresponding Cas protein if the sequence of the associated CRISPR locus is known.

A CRISPR repeat sequence disclosed herein can be a CRISPR repeat sequence for a Cas protein that is capable of processing its own pre-crRNA in to mature crRNA (i.e. processing natural arrays without tracrRNA), for example Cas 12a (Cpf1) or Cas13a (C2c2). For example, the repeat sequence can be for FnCpf1, AsCpf1, or LbCpf1.

A CRISPR repeat sequence can include about 15 to about 36 nucleotides (e.g. about 16, 17, 18, 19, 20, 21, 22, 23, 24, 25, 26, 27, 28, 29, 30, 31, 32, 33, 34, or 35 nucleotides). In some embodiments the CRISPR repeat sequence can include about 20 to about 36 nucleotides, about 25 to about 36 nucleotides, about 30 to about 36 nucleotides, about 15 to about 25 nucleotides, or about 20 to about 25 nucleotides.

A spacer sequence can include any desired nucleic acid sequence within a target gene. For example, the first spacer sequence can include a first target site in a target gene, and the second spacer sequence can include a second target site in the target gene. In some instances, the first spacer sequence includes a target site in a first target gene, and the second spacer sequence includes a target site in a second target gene. Each of the first portion of the first spacer sequence, the second portion of the first spacer sequence, and the first portion of the second spacer sequence can include about 5 to about 20 nucleotides (e.g. about 6, 7, 8, 9, 10, 11, 12, 13, 14, 15, 16, 17, 18, or 19 nucleotides).

Each of the first and second oligonucleotides can include about 40 to about 70 nucleotides (e.g. about 41, 42, 43, 44, 45, 46, 47, 48, 49, 50, 51, 52, 53, 54, 55, 56, 57, 58, 59, 60, 61, 62, 63, 64, 65, 66, 67, 68, or 69 nucleotides). In some embodiments, each of the first and second oligonucleotides can include about 55 to about 65 nucleotides, about 60 to about 65 nucleotides, or about 55 to about 60 nucleotides. In some instances, the first and/or second oligonucleotide are phosphorylated at the 5′ end. The length of the bridge oligonucleotide can be about 30 to about 50 nucleotides (e.g. 31, 32, 33, 34, 35, 36, 37, 38, 39, 40, 41, 42, 43, 44, 45, 46, 47, 48, or 49 nucleotides).

### II. Methods of Generating CRISPR Arrays

The presently disclosed methods of generating CRISPR arrays generally include providing a first and a second oligonucleotide, and a bridge oligonucleotide. The first oligonucleotide, the second oligonucleotide and the bridge oligonucleotide are hybridized together to form a complex. Forming such a complex positions the first and second oligonucleotides in close proximity to facilitate ligation.

Prior to hybridization, the methods described herein can include phosphorylating the first and/or second oligonucleotides, for example, by using T4 polynucleotide kinase. Phosphorylating can occur at about 25° C. to about 45° C. (e.g., about 30° C. to about 40° C., about 35° C. to about 40° C., or about 37° C.).

Hybridization of the first oligonucleotide, the second oligonucleotide, and the bridge oligonucleotide can be performed in a solution. When hybridizing in solution, the concentration of the first oligonucleotide can be, e.g., about equal to a concentration of the second oligonucleotide. Depending upon the methods and oligonucleotides employed, the concentration of the bridge oligonucleotide in the solution may be about equal to, more than, or less than, a concentration of the first oligonucleotide in the solution, or a concentration of the second oligonucleotide in the solution. For example, the concentration of the bridge oligonucleotide, the first oligonucleotide, and the second oligonucleotide can be about equal. In some instances, the bridge oligonucleotide is used at a ratio of between about 2:1 and about 3:1 by molarity in relation to a mixture of the first and second oligonucleotides.

In some instances, hybridization comprises heating the solution to a temperature of about 70° C. to about 100° C. (e.g. about 75° C. to about 95° C., about 80° C. to about 90° C., or about 85° C.). Hybridization can further include cooling the solution to a temperature of about 25° C. to about 45° C. (e.g. about 30° C. to about 40° C., about 35° C. to about 40° C., or about 37° C.) after heating. For example, hybridization can include cooling the solution to about 37° C. after heating the solution to about 85° C. Hybridization can include cooling the solution to a temperature at which a ligase used in the presently described methods retains ligase activity sufficient to ligate the first and second oligonucleotides. In some instances, annealing does not include heating the solution. Depending on the specific method being performed, cooling the solution after heating can include reducing the temperature of the solution at a constant rate or at an uncontrolled rate. For example, hybridization can include heating the solution to about 85° C. followed by cooling the solution to about 37° C. at 0.1° C. per second.

In general, ligating the first and second oligonucleotides can be carried out at a temperature of about 25° C. to about 45° C. (e.g., about 30° C. to about 40° C., about 35° C. to about 40° C., or about 37° C.). Ligating the first and second oligonucleotides can be carried out for various time periods depending on the method being performed, e.g., for about 0.1 to about 48 hours, e.g., about 0.3 to about 45 hours, about 0.5 to about 40 hours, about 0.7 to about 35 hours, about 1 to about 30 hours, about 1.5 to about 25 hours (e.g., about 1, 2, 4, 6, 8, 10, 12, 14, 16, 18, 20, 25, 30, 35, 40, or about 45 hours).

A variety of ligases may be used in the presently described methods. For example, the ligase can be a T4 DNA ligase, T3 DNA ligase, T7 DNA ligase, Taq DNA ligase, PBCV-1 DNA ligase, thermostable DNA ligase (e.g., 5′AppDNA/RNA ligase), or an ATP dependent DNA ligase. Combinations of any two or more such ligases may be used in some instances.

In some methods described herein, three or more (e.g., 4, 5, 6, 7, 8, 9, or 10 or more) oligonucleotides can be ligated to generate a CRISPR array. Ligation of the three or more oligonucleotides can be carried out in the same step, or in separate steps (such as in a step-wise fashion).

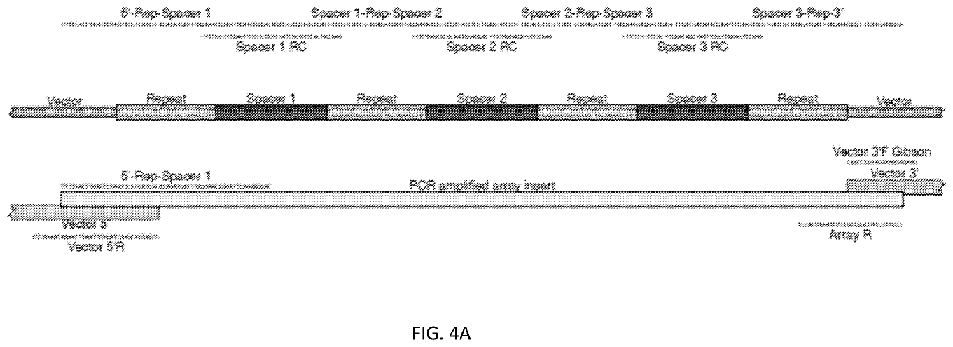

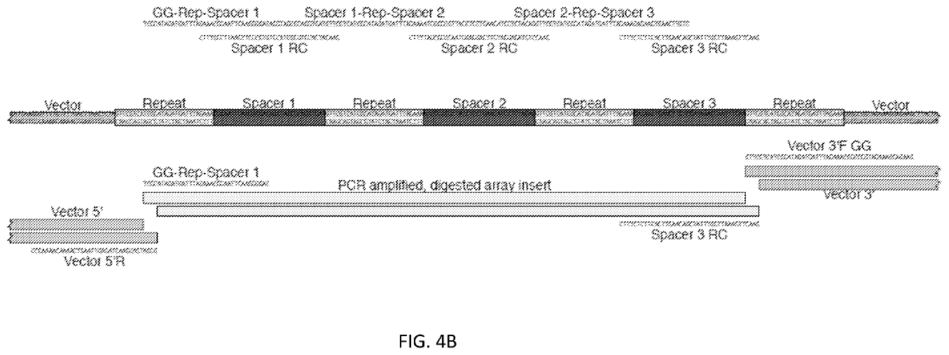

FIG. 4 is a schematic diagram showing the ligation of four oligonucleotides using three bridge oligonucleotides. By way of illustration, a CRISPR array can be generated by ligating oligonucleotides 5′-Rep-Spacer 1, Spacer 1-Rep-Spacer 2, Spacer 2-Rep-Spacer 3, and Spacer 3-Rep-3′ (listed in a 5′ to 3′ order), using bridge oligonucleotides Spacer 1 RC, Spacer 2 RC, and Spacer 3 RC. The first and second oligonucleotide described herein can be 5′-Rep-Spacer 1 and Spacer 1-Rep-Spacer 2, respectively; while the bridge oligonucleotide can be Spacer 1 RC. The first and second oligonucleotide described herein can also be Spacer 1-Rep-Spacer 2 and Spacer 2-Rep-Spacer 3, respectively; while the bridge oligonucleotide is Spacer 2 RC. In some embodiments, the methods described herein can also include ligating an oligonucleotide at the 3′ end of the array, where the oligonucleotide includes a portion of the last spacer sequence of the array at the 3′ end, a CRISPR repeat sequence or a portion thereof, and a flanking sequence. The flanking sequence can include a portion of the sequence of a vector. For example, Spacer 3-Rep-3′ as shown in FIG. 4 includes, from 5′ to 3′, a portion of Spacer 3, a CRISPR repeat, and a portion of the sequence of a vector.

Methods described herein can further include purifying the ligation product to remove unligated oligonucleotides. Purification can include, for example, the use of a PCR purification column. The methods can further include generating a strand complementary to the ligated first and second oligonucleotide, wherein the complementary strand comprises the bride oligonucleotide, thereby generating a double-strand construct. The double-strand construct can be further purified. Purification can include the use of a PCR purification kit (any suitable kit known in the art), or running the double-strand construct on a gel followed by purification of the DNA using a gel extraction kit (any suitable gel extract kits known in the art). The methods can further include inserting the CRISPR array into a vector. Various methods for cloning PCR products into a vector are known in the art, for example, Gibson Assembly or Golden Gate cloning. Any suitable vectors or plasmids known in the art can be used for inserting the CRISPR array and subsequent transformation into host cells to generate clones that carry the CRISPR arrays. In some embodiments, pBAV lk can be used.

Vectors comprising CRISPR arrays generated using methods described herein are also contemplated by the present disclosure.

### III. Cas Proteins

The presently disclosed methods of generating a CRISPR array include providing a first oligonucleotide and a second oligonucleotide, where the first oligonucleotide, the second oligonucleotide, or both, comprises a CRISPR repeat sequence or a portion thereof that can bind to a Cas protein.

The Cas protein can be naturally-occurring or non-naturally occurring. Examples of such Cas proteins include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cpf1 (also known as Cas 12a), Cas13a (C2c2) and functional derivatives thereof. The Cas protein can be a small Cas protein. The small Cas proteins can be engineered from portions of Cas proteins derived from any of the Cas proteins described herein and known in the art. In some cases, a small RNA-guided nuclease is, e.g., smaller than about 1,100 amino acids in length.

The Cas protein can be a mutant Cas protein, e.g., a mutant of a naturally occurring Cas. The mutant Cas can have altered activity compared to a naturally occurring Cas, such as altered endonuclease activity (e.g., altered or abrogated DNA endonuclease activity without substantially diminished binding affinity to DNA). Such modification can allow for the sequence-specific DNA targeting of the mutant Cas for the purpose of transcriptional modulation (e.g., activation or repression); epigenetic modification or chromatin modification by methylation, demethylation, acetylation or deacetylation, or any other modifications of DNA binding and/or DNA-modifying proteins known in the art. In some instances, the mutant Cas has no DNA endonuclease activity.

The Cas protein can be a nickase that cleaves the complementary strand of the target DNA but has reduced ability to cleave the non-complementary strand of the target DNA, or that cleaves the non-complementary strand of the target DNA but has reduced ability to cleave the complementary strand of the target DNA. In some instances, the Cas protein has a reduced ability to cleave both the complementary and the non-complementary strands of the target DNA.

### EXAMPLES

### Example 1: Construction of Multiplex CRISPR Arrays

Described here is a technique that can accurately assemble a multiplex natural CRISPR array in just 1 day. The technique requires no sequence modifications and uses only standard-length DNA oligos. This strategy was used to assemble multiplex CRISPR arrays of up to 9 spacers and demonstrated in bacteria, including arrays from both a Type I-F CRISPR system and a Cas12a system.

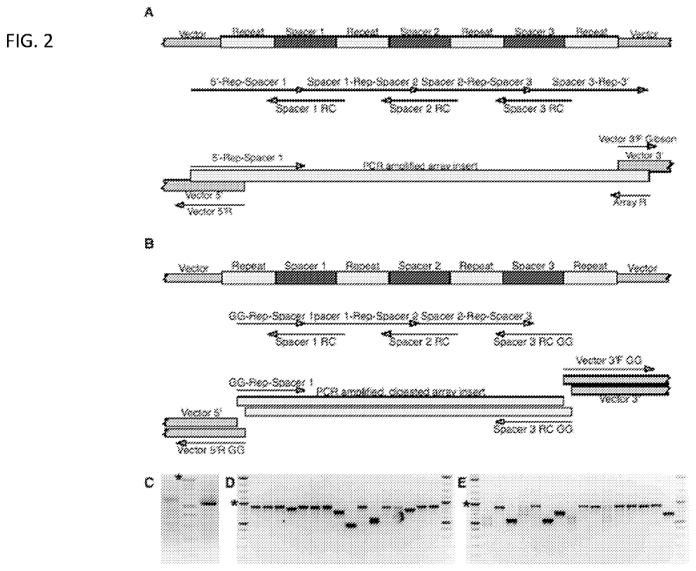

An insight of the method is that it assembles only the top strand of the array using ligation, and then later fills in the bottom strand using PCR (FIG. 2B). During annealing and ligation, the top strand oligos are joined by shorter bottom oligos that only cover the spacer regions. This restricts ligation junctions to the unique spacer regions of the array, while leaving single-stranded gaps that cover the repeat portions of the array. In this way, the method avoids incorrect annealing, ligation junctions, or spacer order, which could otherwise result from annealing between repeat regions.

A. baylyi Contains a Functional Type I-F CRISPR-Cas System

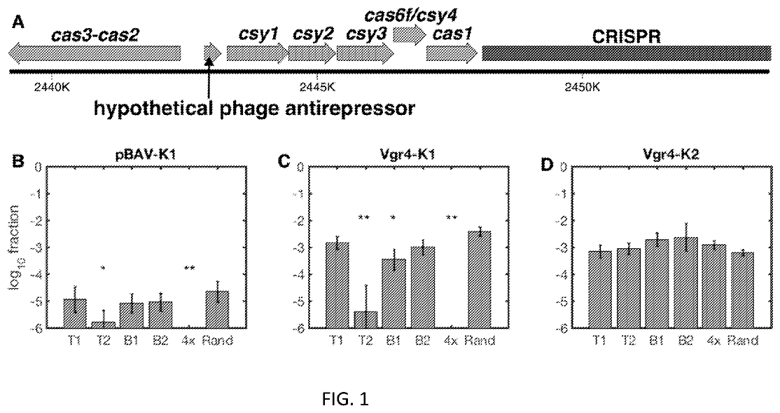

The A. baylyi genome contains a computationally identified Type I-F CRISPR-Cas system (FIG. 1A), but its function has not been tested experimentally. Therefore, we first determined whether the endogenous CRISPR-Cas system can block horizontal gene transfer via natural competence. To test the system, we inserted single-spacer arrays targeting a kanamycin resistance gene into a previously used neutral locus in the genome. We tested four different spacers from both the top (T) and bottom (B) strands, each using the 5′-CC-protospacer-3′ protospacer-adjacent motif (PAM, 5′-anti-protospacer-GG-3′ on the complementary, targeted strand) previously shown to work in the Type I-F systems of E. coli, Pectobacterium astrosepticum, and Pseudomonas aeruginosa⁴¹. When naturally competent cells carrying these single arrays were incubated with a self-replicating plasmid (pBAV-K1), there were still many kanamycin-resistant transformants, and only the T2 spacer reduced the transformation efficiency relative to a random spacer (FIG. 1B, note the log scale). When they were challenged using a genomically integrating linear DNA construct (Vgr4-K1), again the T2 spacer worked well, now decreasing acquisition of kanamycin resistance by 1000-fold relative to a random spacer, but the others were less effective (FIG. 1C). Escape clones did have somewhat smaller colony sizes, suggesting partial tolerance for ongoing self-targeting. All strains remained competent for Vgr4-K2, which contains a second kanamycin resistance gene with minimal homology to the first (FIG. 1D).

### Construction of Multiplex CRISPR Arrays

To increase the efficacy of the endogenous A. baylyi CRISPR-Cas system against incoming DNA, multiplex arrays were developed, which have been reported to increase CRISPR efficacy in a variety of contexts. However, constructing natural, multiplex Type I CRISPR arrays remains challenging for the reasons described above. Therefore, a new method was developed to assemble multiplex, completely natural arrays.

This method is based on annealing and ligating single-stranded DNA oligos (FIG. 2). An insight is that despite extensive repetition, the correct order can be ensured by avoiding annealing or ligation within repeats. To achieve this, 60 nt top oligos were designed that each include a single 28 nt repeat in their center and extend halfway (16 nt) into the spacer or flanking sequence on either side. These top oligos are joined together by annealing to 40 nt bottom bridge oligos, consisting of the reverse complement of each 32 nt spacer plus 4 nt of repeat on either side. The intentional gaps on the bottom strand avoid oligo annealing within repeats, and they are filled in later by PCR. Multiple conditions were tested to optimize the assembly protocol (FIG. 2).

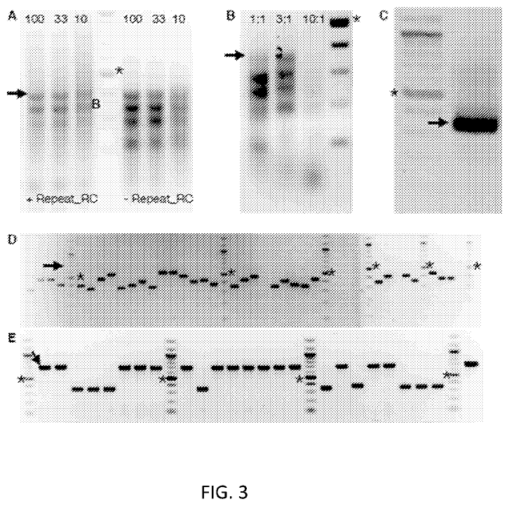

Protocol optimizations were performed using a 6×IS-CRA array and inserted into pBAV using Golden Gate assembly. An oligo covering the remaining 20 nt of the repeats to fill in the gaps on the bottom strand (repeat_RC) was tested, but this resulted in a smear of larger than expected ligation products, indicating increased ligation at incorrect junctions (FIG. 3). Furthermore, while developing this protocol several correct-sized clones were sequenced that had incorrect spacer order, but only when including the repeat_RC oligo. FIGS. 3A and B: raw ligations; FIG. 3C: PCR amplification; FIGS. 3D and E: Colony PCR screening of clones. Asterisks on all gels indicate the 500 bp band of the ladder, and arrows indicate the correctly sized assembly. As shown in FIG. 3A, including the Repeat_RC oligo increases incorrect, higher-molecular-weight smearing (left 3 vs right 3 lanes), and 100 μM stock oligos (lanes 1 and 5) work better than 33 μM (lanes 2 and 6) or 10 μM (lanes 3 and 7) stock solutions. The center (lane 4) is a 100 bp ladder. As shown in FIG. 3B, annealing and ligation is most efficient using 3 parts bottom oligos to 1 part top oligos. The lanes from left to right are ligations using 1:1, 3:1, and 10:1 ratios of bottom oligos to top oligos, followed by a 100 bp ladder. As shown in FIG. 3C, PCR amplification of the resulting ligation improves yield of the correct sized product. As shown in FIG. 3D, Golden Gate assembly directly from ligation products yielded no correct-sized arrays out of 36 tested clones. All of 6 sequenced clones were correct at the 3′ end, but truncated at the 5′ end of the array. FIG. 3E shows that, as for FIG. 3D, but the ligation product was PCR amplified and gel extracted before inserting into the vector. 16 of 25 colonies were the correct size, and all incorrect clones had Ox arrays (a single repeat only).

An example protocol, by way of illustration only, is as follows:

1. Phosphorylation: Mix 2 to 4 μl of each top oligo from 100 μM stock solutions (FIG. 4A), and phosphorylate them using T4 polynucleotide kinase (PNK) and 1×T4 DNA ligase buffer at 37° C. for 15-60 minutes. This step can be skipped if ordering 5′ phosphorylated oligos. Phosphorylating the top oligos separately increases PNK activity, which is optimal on single-stranded DNA.

2. Annealing: Mix 1 part top oligos with 2-3 parts bottom oligos by molarity (FIG. 4B), and perform a slow annealing starting from 90° C. We used a thermocycler programmed to decrease to 37° C. by 0.1° C./sec, but allowing a hot water bath to gradually cool should work as well.

3. Ligation: Add T4 DNA ligase and additional ligase buffer, and incubate at 37° C. for 30 minutes.

4. Clean up: Column purify the ligated array using a standard DNA purification column to remove unincorporated oligos.

5. Amplification: PCR amplify the array using primers appropriate for your cloning strategy of choice, e.g., Gibson or Golden Gate assembly, using as high an annealing temperature as the primers will allow (FIGS. 3C-E).

6. OPTIONAL: Gel Purification: Run the raw ligation or amplified PCR product on an agarose gel, excise the correct band, and purify the DNA using a gel extraction kit. This step is optional for shorter arrays, but it can substantially increase accuracy for longer arrays.

7. Insert into vector: Insert the array into a vector, e.g., Golden Gate, Gibson assembly, or fusion PCR.

8. Transform: Transform the final construct into E. coli (for circular plasmids), or directly into A. baylyi (for linear constructs with genomic homology for recombination), spread on selective agar plates, and incubate overnight.

9. (Next day) Screen: On the following day, pick several colonies and PCR across the array to screen for assemblies of the correct length (FIGS. 3D, E).

The assembly steps can be completed in one day, and the resulting colonies can be screened the following day by PCR across the CRISPR array. This basic array assembly technique is compatible with multiple cloning strategies for insertion into a final vector. In developing our protocol, we successfully inserted the arrays into circular plasmids using both Gibson (FIGS. 2A and 4A) and Golden Gate (FIGS. 2B and 4B) cloning strategies, as well as into linear DNA fragments that we amplified via PCR.

Using our optimized protocol, we were able to quickly and accurately assemble a 9-spacer array (FIG. 2C), using either Gibson or Golden Gate strategies to insert the array into the plasmid. For Golden Gate insertion, 11 of 16 picked colonies had the correct length array (FIG. 2D), and for Gibson insertion, 8 of 16 picked colonies had the correct length (FIG. 2E). Sanger sequencing confirmed that all arrays with the correct length were assembled in the correct order. 7 of the Golden Gate and 2 of the Gibson clones were completely correct, and the remainder had various indels or substitutions. Only one of the errors was at a junction between oligos, suggesting most may have occurred during oligo synthesis.

### Multiplex Natural Arrays Enhance CRISPR Efficacy in Natural Competence

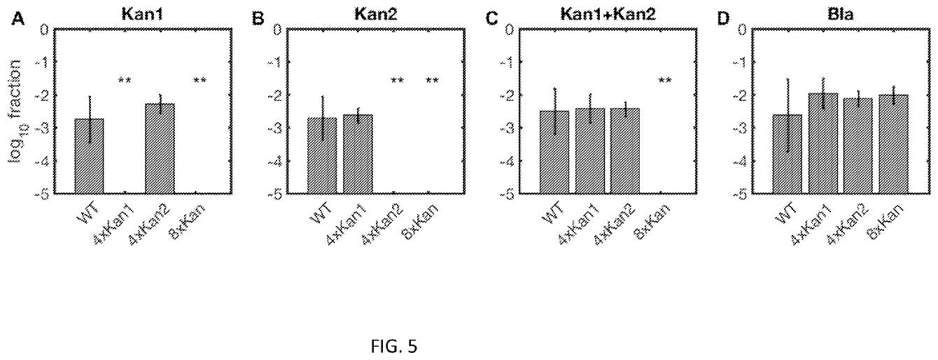

To see if multiplex CRIPSR arrays more effectively interfere with natural competence in A. baylyi, the 4 spacers targeting the kanamycin resistance gene were combined into a single, 4-spacer natural array and inserted it into the A. baylyi genome. This 4×Kan1 array was highly effective against both the self-replicating plasmid pBAV-K1 and the genomically integrating construct Vgr4-K1 (FIGS. 1B, C, FIG. 5A). As for single-spacer arrays, the 4-spacer array was ineffective against a second, control kanamycin resistance gene with no homology to the targeted gene (FIG. 1D, FIG. 5B). The 4-spacer array allowed no escape transformants with the replicating plasmid, but we did obtain 2 escapes with the integrating construct. In one of these escapes, the inserted 4-spacer CRISPR array had been disrupted by the active insertion sequence IS1236. The other escape appeared to have a larger genomic deletion encompassing the array, as it had lost the spectinomycin resistance marker used to select for insertion of the array, and the entire region failed to amplify by PCR.

Next, we expanded our array to defend against both kanamycin resistance genes simultaneously, using an 8-spacer array. As a preliminary step, a 4-spacer array was constructed targeting the second kanamycin gene, added genomic homology arms via fusion PCR, and cloned the linear product into A. baylyi. Then an 8-spacer array was assembled targeting both kanamycin resistance genes. This 8-spacer array was assembled in a one-pot reaction, but we also assembled it from the individual 4-spacer arrays to demonstrate modular array construction. For the modular approach, a cloned 4×Kan2 array was PCR amplified using a leftmost top primer that began with the first 16 bp of the final spacer in the 4×Kan1 array rather than with the 5′ region of the vector, and then performed a fusion PCR of the 3 pieces Vector 5′-4×Kan1, 4×Kan2, and Vector 3′.

In contrast to single spacers (FIG. 1), each 4-spacer array effectively blocked acquisition of its respective kanamycin resistance gene (FIGS. 5A, B), and only the 8-spacer array prevented acquisition of kanamycin resistance when both genes were present (FIG. 5C). All arrays allowed acquisition of a non-homologous beta-lactamase gene (FIG. 5D). The modular construction shows that even if there is a size limit to this method, very large arrays can still be assembled in very few steps.

### Markerless Genome Editing Using An Endogenous CRISPR-Cas System

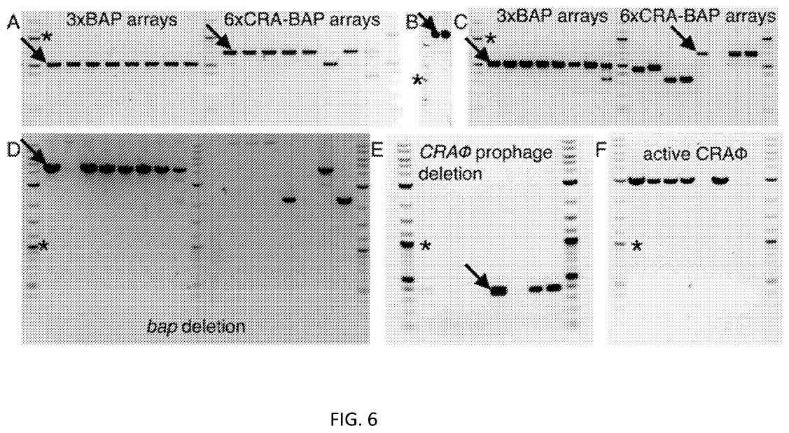

CRISPR has been used for genome editing in many contexts, and we wanted to confirm that our natural arrays would enable editing of the A. baylyi genome as well. To do this, a 3-spacer array targeting the bap gene (ACIAD2866) was constructed, which has been implicated in biofilm formation in Acinetobacter, and thus may be at least partially responsible for intractable clogging when using A. baylyi in microfluidics. The 3×BAP array was inserted into both pBAV1spec for cloning into E. coli, as well as into a linear construct with roughly 1 kb genomic homologies on either side for direct insertion into the A. baylyi genome. The pBAV1spec assembly transformed into E. coli was the correct length in 8 of 8 tested clones (FIG. 6A, left half). Four were sequenced, of which all had the correct spacer order, although one was missing two base-pairs. When this pBAV1spec-CRISPR_{3×BAP} was co-transformed into A. baylyi along with a markerless bap deletion donor DNA (linear dsDNA with ˜1 kb homology arms on either side), both of two tested clones had the correct deletion (FIG. 6B). Interestingly, the bap in our strain of A. baylyi ADP1 (ATCC 33305) was approximately 3 kb larger than in the published genome. This may have been due to a sequence assembly error or genomic instability, either of which could result from the many tandem repeats found in bap genes.

When using a linear construct to deliver the 3×BAP array into A. baylyi, many more clones were obtained than when using pBAV1spec (on the order of 1000 vs 36), which is expected because homologous recombination is more efficient than plasmid re-circularization in A. baylyi natural competence. Of 8 tested clones, 7 had the correct size array (FIG. 6C, left half) and 7 had the correct BAP deletion (FIG. 6D, left half), even despite the CRISPR array not having first been clonally verified.

Next, a 6×array targeting both bap and the CRAΦ prophage was created by deleting two genes, which binds the competence machinery when activated, complicating horizontal gene transfer experiments. The pBAV1spec-CRISPR_{6×CRA-BAP} construct had the correct array length in 6 of 8 E. coli clones (FIG. 6A, right half), but no double genomic deletion in A. baylyi, likely due to the relative inefficiency of circular plasmids in natural transformation.

To increase transformation efficiency, the genomically integrating, linear 6×CRA-BAP construct was used along with CRAΦ and bap deletion donor DNAs. Of 8 tested clones, 3 had the correct array length (FIG. 6C, right half). All 3 of those had both the desired genomic CRAΦ deletion (FIG. 6E) and eliminated the excised, circular CRAΦ episome (FIG. 6F). All three clones also had mutations in bap, although two of them had larger deletions (FIG. 6D, right half), leaving one clone with both precise deletions. One of the larger bap deletions extended to the end of a nearby copy of the insertion sequence IS1236, and the other had a more complex rearrangement that appeared to involve an inversion of part of the genome. IS1236 is not present next to bap in the official genome sequence, but it was already there in our parental strain before the double deletion attempt. This is not completely unexpected, since IS1236 is known to be highly active in A. baylyi. If the correct editing rate were more important than speed, one could likely increase the percentage of clones with the correct edits by first clonally verifying the linear CRISPR array construct.

### Construction of Cas12a Arrays

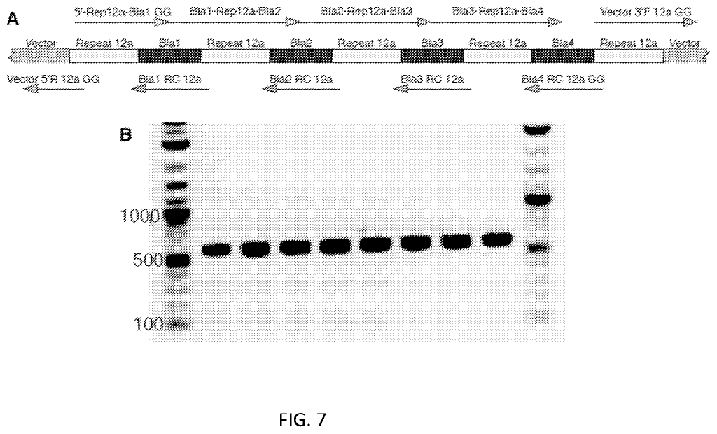

In some embodiments, the method described here is generalizable to other natural CRISPR arrays, which use different repeat sequences and spacer lengths. For this demonstration, Cas12a/Cpf1 arrays were chosen, which are processed by their respective single effector nuclease. The Cas12a CRISPR array unit for Franciscella novicida U112 is slightly longer than the A. baylyi array unit, with 36 bp repeats and 26-32 bp spacers. Nevertheless, a 4-spacer array with a full 68 bp unit length was assembled, targeting a beta lactamase gene (FIG. 7A). All screened clones (8 of 8) had the full-length array in the correct order (FIG. 7B) of which 2 were correct with no gaps.

The method presented here solves the challenge of rapid, affordable, and scalable construction of completely natural multiplex CRISPR arrays, with no sequence modifications and only minimal constraints. This should be highly beneficial for multiple applications in a variety of organisms, from basic research to applied tools. For applications using heterologous, array-processing Cas nucleases such as Cas12a, facile construction of multiplex natural arrays will help with gene regulation, genome engineering, and even population engineering.

This assembly method includes at least 3 key features that improve its accuracy and efficiency: unique ligation junctions, long annealing regions, and limited oligo length. In the first feature, the only ligation junctions are within the unique spacers on the top strand, which helps to ensure assembly in the correct order. Gaps were left in the repeat regions on the bottom strand to avoid ligation junctions within repeats. We tested including an oligo covering the remaining 20 nt of the repeats to fill in the gaps on the bottom strand (repeat_RC), but this resulted in a smear of larger than expected ligation products, indicating increased ligation at incorrect junctions (FIG. 3A). Furthermore, while developing this protocol several correct-sized clones were sequenced that had incorrect spacer order, but only when including the repeat_RC oligo.

The second feature is long (20 nt) annealing regions that allow more rapid and specific annealing and ligation than the usual 4 bp Golden Gate overlaps, particularly at the 37° C. where T4 DNA ligase has optimal activity. The long annealing regions also allow the user to choose spacers without constraints imposed by the requirement for junction orthogonality, since such long sequences should be highly specific. This allows for very easy, plug-and-play oligo design. Third, the longest oligos must only be the unit length of the CRISPR array, which for A. baylyi is 60 nt. Oligos of this length are relatively reliable, affordable, and rapidly delivered from most DNA synthesis vendors.

A further advantage lies in cost-saving oligo reusability. Unlike ad-hoc construction strategies, this method places the ligation junctions in the same location for every spacer-repeat unit, meaning that many oligos can be reused for alternate array designs without checking for compatibility. For example, our 4×Kan1 and 4×Kan2 arrays were easily joined with just one additional oligo. This modular assembly demonstrates that verified sub-arrays can easily be joined with just one additional day of work.

The PCR amplification step following ligation both enriches the correct size product and produces a double-stranded construct with no gaps. A fully double-stranded insert is important for Gibson Assembly-based insertion into the vector because of the required exonuclease, but also important for Golden Gate insertion. Without PCR amplification, Golden Gate insertion of a 6×array yielded clones containing a range of incorrectly sized inserts (compare FIGS. 3D and 3E). Interestingly, these incorrect arrays almost always contained spacers that were in the correct order, but truncated at the 5′ end. The 5′-specific truncation may involve a gap repair process within the E. colihost that may be mediated by repeats and directionally biased by plasmid replication.

In prokaryotes with endogenous CRISPR-Cas systems, this method will improve the study and understanding of the ecological importance of CRISPR in its natural context, including the antagonistic interplay between CRISPR and horizontal gene transfer (HGT). This seemingly contradictory pair of abilities has raised evolutionary questions about tradeoffs between the acquisition of new traits via HGT, versus CRISPR-mediated exclusion of foreign DNA. This interaction is important for microbial evolutionary theory, but when the transferring genes confer antibiotic resistance or pathogenicity, it also directly impacts human health. Here, in the highly competent A. baylyi, the CRISPR-HGT interaction is not straightforward. While multiplex arrays effectively blocked exogenous DNA uptake, weaker single spacers reduced, but did not eliminate, HGT. This suggests that for A. baylyi, one solution to the CRISPR-HGT conundrum is to hedge their bets. Single spacers provide some protection against incoming targeted DNA, but particularly for weaker spacers or when multiple spacers compete for limited CASCADE complexes, some targeted DNA can still be acquired. When the tolerance is only partial, the targeted protospacer (or the CRISPR machinery) will eventually mutate to eliminate genomic self-targeting and alleviate growth costs, allowing ongoing exploration of the genetic diversity in the environment.

### Example 2: Methods Used in the Above Experiment

### Array Construction

Spacers were designed to match target sequences preceded by CC on the non-targeted strand using a computational tool to ensure they were maximally orthogonal to the rest of A. baylyi genome. Briefly, the algorithm searches for all possible spacers in the target sequence that have the appropriate PAM, and then scans them against the host genome to find the most similar sequence, giving greater weight to bases in the PAM-proximal seed sequence. The best match (highest score) against the host genome is assigned as the score for that spacer. Spacers were chosen from among the lowest scoring (most genome-orthogonal) sequences to cover the entire target and include both DNA strands. For a random spacer, the lowest scoring sequence was selected among a computer-generated, random pool. Oligos were designed according to the diagrams in FIG. 2 and FIG. 4, and their sequences are given in Table 1. Spacer sequences are shown in Table 2. Standard quality, desalted oligos normalized to 100 μM in TE buffer from ValueGene, Eton Bio, and Integrated DNA Technologies were used. All enzymes and buffers were from New England Biolabs. An example protocol, by way of illustration only, is as follows:

1. Phosphorylate oligos by mixing 1-2 μl of each top-strand oligo along with 1×T4 ligase buffer and 1 μl T4 polynucleotide kinase (NEB). Polynucleotide kinase buffer will not work without supplementary ATP. Incubate at 37 degrees for 30-60 minutes.

2. Anneal oligos by mixing 1 part phosphorylated top oligos with 2 to 3 parts bottom oligos, heating to 85° C., and slowly cooling back to 37° C. at 0.1° C. per second in a thermocycler.

3. Ligate by adding 1 μl T4 DNA ligase and another 1×ligase buffer. Incubate at 37° C. for another 30-60 minutes.

4. Remove unligated oligos using a PCR purification column (Lamda Biotech).

5. PCR amplify the ligation product using primers as shown in FIGS. 2 and Table 1. We used Q5 DNA polymerase and the manufacturer's recommended protocol, annealing at 72° C., extending for 20 seconds, and running for 20 cycles. A high annealing temperature is critical to recover the correct product; primers can be checked using commonly available software.

6. Purify the PCR product either directly or after excising the correct band from a gel, using a column-based PCR or gel purification kit (Qiagen).

7. Insert the array into a vector. For Gibson assembly, we mixed 2 μl total DNA (with equimolar parts) with 2 μl of 2×master mix and incubated at 50° C. for one hour. For Golden Gate assembly, we mixed 4 μl total DNA (with equimolar parts), 0.5 μl T4 DNA ligase buffer, 0.25 μl T4 DNA ligase, and 0.25 μl BsaI, and incubated for 30-50 cycles of 1 minute each at 37° C. and 24° C., followed by 10 minutes at 50° C. Vectors were prepared by PCR using primers as shown in FIGS. 1 and S2, and gel extracted. Whenever the vector PCR was derived from a plasmid, we used the primers Vector 3′F and Vector 5′R and treated the product with DpnI. For linear constructs used in direct transformation into A. baylyi, the vector consisted of approximately 1 kb homology arms on either side of the array. In these cases, we either directly mixed the 3 pieces (5′ arm, array, and 3′ arm) in a full-length PCR reaction, or first pre-joined the 3 pieces via either Gibson or Golden Gate assembly, and then PCR amplified and gel extracted the full construct.

For modular assembly of the 8×Kan array, both 4×Kan1 and 4×Kan2 arrays were assembled and inserted into the genomic integration vector as above. Next, the 5′ part of the 4×Kan1 construct was PCR amplified through the array using the primers pp_5′F and Kan1_B2_RC, as well as the 4×Kan2 construct using the primers Kan1_B2-R-Kan2_T1 and Array_R. Then 3-piece PCR with primers Vector_5′F and Vector_3′R were used to fuse (i) Vector 5′-4×Kan1, (ii) 4×Kan2, and (iii) the vector 3′ piece (amplified using primers Vector_3′F and pp_3′R).

To assemble FnCas12a arrays, the same procedure described above was followed, using the Golden Gate insertion strategy.

### Cell Culture, Transformations, and Screening

All cells were grown in LB media at 30 or 37° C. A. baylyi strain ADP1 was obtained from ATCC (stock #33305) and for E. coli a lab strain of MG1655 was used. The kan1 gene was aminoglycoside O-phosphotransferase APH(3′)-IIIa, and the kan2 gene was aminoglycoside O-phosphotransferase APH(3′)-IIa. These two genes have no significant similarity as determined by BLAST alignment. For transformation of A. baylyi via natural competence, cultures were washed overnight, resuspended in fresh LB, and incubated 50 μl of cells plus DNA at 37° C. for 2 to 4 hours. All data plotted in the same figure used the same concentration of donor DNA, generally 0.2-1 ng/μl. To quantify the fraction of transformed cells, we performed five 10-fold serial dilutions and spotted 3 measurement replicates of 2 μl each at each dilution level onto 2% agar LB plates containing the appropriate (or no) antibiotic selection (20 μg/ml of kanamycin and/or spectinomycin). Each experiment was repeated on two separate days. Lower agar concentrations did not work well for colony counting, because the motile cells began to spread and colonies became less well-defined. Only colonies visible after 20 hours at 30° C. for 20 hours were counted.

CRISPR arrays were inserted into a neutral genomic region that has been used previously, replacing genomic coordinates 2,159,575-2,161,720, covering ACIAD2187, ACIAD2186 and part of ACIAD2185. The integration site for CRISPR-targeted kanamycin resistance genes was another region found to be neutral in our lab conditions, ACIAD3427. The upstream homology arm covered coordinates 3,341,420-3,342,480, and the downstream homology arm covered 3,342,641-3,343,720. The replicating plasmid was the broad host pBAV1k, which was modified to spectinomycin resistance when using it to carry CRISPR arrays. In arrays, the 80 bp upstream of the endogenous CRISPR array was included to include any leader sequences or regulatory elements. For markerless genomic deletions, a linear donor DNA was constructed by PCR fusing approximately 1 kb regions upstream and downstream of the targeted gene.

For PCR screening of clonal CRISPR arrays in E. coli, individual colonies were selected into 50 μl of water, and used 1 μl directly in a PCR reaction. A. baylyi did not obtain clean results unless a genomic miniprep kit was first used to purify DNA (Promega Wizard). Colors were inverted for all agarose gels to assist visualization.

### Statistical Analysis

To calculate error bars for ratios on logarithmic plots, error propagation was used as described previously. For each experimental replicate (each with 3 measurement replicates; i.e., 2 μl spots), we took the log base 10 of each data point, found the standard deviations for both transformed and total cell count measurement replicates (σ₁ and σ₂), and calculated the standard deviation of the ratio as a σ=\sqrt{σ₁²+σ₂²}. To find the total variance across experimental replicates from different days, we used the error propagation formula

$${σ}^{2} = \frac{{\sum }_{c} [ ( {n}_{c} - 1 ) {{σ}_{c}}^{2} + {{n}_{c} ( {f}_{c} - f )}^{2} ]}{( {\sum }_{c} {n}_{c} ) - 1} ,$$

where the subscript c denotes experimental replicates, f is the fraction transformed, and n_{c} is the number of measurement replicates for each experiment (here, 3 spotting replicates). Performing calculations on a logarithmic scale creates a problem when some, but not all, measurement replicates are below the limit of detection, because zeros create infinities. In these cases, we set the zeros to half the limit of detection as a conservative estimate for the purposes of plotting, since excluding them would artificially increase the average for that experiment.

We performed significance tests as described previously. In FIGS. 1 and 3, we performed multiple comparison tests using the Matlab function multcompare, using the error propagated means and variances (on log 10 scales) and Tukey's HSD criterion. Where data was below the limit of detection, we tested for difference from that limit of detection.

TABLE 1

| Purpose | Name | Sequence |
|---|---|---|
| CRISPR_{4xKan1} | kan1 5′-R-T1 | TTTTGACTTAACTCTAGTTCGTCATCGCATAGATG |
| Gibson | | ATTTAGAAAGGTCGATCAGGGAGGA |
| | kan1 T1-R-B1 | TATCGGGGAAGAACAGGTTCGTCATCGCATAGAT |
| | | GATTTAGAAATTGCATTCTAAAACCT |
| | kan1 B1-R-T2 | TAAATACAGAAAACAGGTTCGTCATCGCATAGAT |
| | | GATTTAGAAAGTCGATACTATGTTAT |
| | kan1 T2-R-B2 | ACGCCAACTTTGAAAAGTTCGTCATCGCATAGATG |
| | | ATTTAGAAAAAGCGAGCTCGGTACT |
| | kan1 B2-R-3′ | AAAACAATTCATCCAGGTTCGTCATCGCATAGATG |
| | | ATTTAGAAACGGCCGGTAGAAAGGA |
| | Kan1 T1 RC | GAACCTGTTCTTCCCCGATATCCTCCCTGATCGAC |
| | | CTTTC |
| | Kan1 B1 RC | GAACCTGTTTTCTGTATTTAAGGTTTTAGAATGCA |
| | | ATTTC |
| | Kan1 T2 RC | GAACTTTTCAAAGTTGGCGTATAACATAGTATCGA |
| | | CTTTC |
| | Kan1 B2 RC | GAACCTGGATGAATTGTTTTAGTACCGAGCTCGCT |
| | | TTTTC |
| CRISPR_{4xKan2} | kan2 5′-R-T1 | TTTTGACTTAACTCTAGTTCGTCATCGCATAGATG |
| Gibson | | ATTTAGAAATCGCCGTCGGGCATGC |
| | kan2 T1-R-B1 | GCGCCTTGAGCCTGGCGTTCGTCATCGCATAGATG |
| | | ATTTAGAAAGGCTACCTGCCCATTC |
| | kan2 B1-R-T2 | GACCACCAAGCGAAACGTTCGTCATCGCATAGAT |
| | | GATTTAGAAACAACCTTACCAGAGGG |
| | kan2 T2-R-B2 | CGCCCCAGCTGGCAATGTTCGTCATCGCATAGATG |
| | | ATTTAGAAAGGCCGCTTGGGTGGAG |
| | kan2 B2-R-3′ | AGGCTATTCGGCTATGGTTCGTCATCGCATAGATG |
| | | ATTTAGAAACGGCCGGTAGAAAGGA |
| | Kan2 T1 RC | GAACGCCAGGCTCAAGGCGCGCATGCCCGACGGC |
| | | GATTTC |
| | Kan2 B1 RC | GAACGTTTCGCTTGGTGGTCGAATGGGCAGGTAGC |
| | | CTTTC |
| | Kan2 T2 RC | GAACATTGCCAGCTGGGGCGCCCTCTGGTAAGGTT |
| | | GTTTC |
| | Kan2 B2 RC | GAACCATAGCCGAATAGCCTCTCCACCCAAGCGG |
| | | CCTTTC |
| Array PCR | Array R | TCCTTTCTACCGGCCGTTTCTAAATCATCT |
| | Array R GG | TTTGGTCTCATCCTTTCTACCGGCCGTTTCTAAATC |
| | | ATCT |
| CRISPR_{8xKan} | Kan1 B2-R- | AAAACAATTCATCCAGGTTCGTCATCGCATAGATG |
| Gibson | Kan2 T1 | ATTTAGAAATCGCCGTCGGGCATGC |
| Vector Gibson | Vector 5′R | TAGAGTTAAGTCAAAACAAAACCC |
| | Vector 3′F | GAAACGGCCGGTAGAAAGGA |
| Vector Golden | Vector 5′R | TTTGGTCTCAGCGATGACGAACTAGAGTTAAGTCA |
| Gate (GG) | GG | AAACAAAACCC |
| | Vector 3′F GG | TTTGGTCTCAGTTCGTCATCGCATAGATGATTTAG |
| | | AAACGGCCGGTAGAAAGGAGAAG |
| Genomic | pp 5′F | TGAGCCGACATTTTATTACCCTCT |
| integrating | | |
| CRISPR vector | | |
| | pp 3′R | TTACCTGAAAGCCAATCGCTG |
| CRISPR_{3xBAP} | GG-R-BAP1 | TTTGGTCTCATCGCATAGATGATTTAGAAACGGAA |
| GG | | TTCAAGGGGAC |
| | BAP1-R- | AGGTAGCGCAGGTGATGTTCGTCATCGCATAGATG |
| | BAP2 | ATTTAGAAAATCGCGCGTTACCTCC |
| | BAP2-R- | TGAACATCCTCTACAGGTTCGTCATCGCATAGATG |
| | BAP3 | ATTTAGAAAGAGAAGTGAACTTGTC |
| | BAP1 RC | GAACATCACCTGCGCTACCTGTCCCCTTGAATTCC |
| | | GTTTC |
| | BAP2 RC | GAACCTGTAGAGGATGTTCAGGAGGTAACGCGCG |
| | | ATTTTC |
| | BAP3 RC GG | TTTGGTCTCAGAACTTGAAATTGGTTTATCGACAA |
| | | GTTCACTTCTCTTTC |
| CRISPR_{3xCRA-3xBAP} | GG-R-CRA1 | TTTGGTCTCATCGCATAGATGATTTAGAAATCTCC |
| GG | | GCGCTTGCTTC |
| | CRA1-R- | GCATAATGCAGATTGAGTTCGTCATCGCATAGATG |
| | CRA2 | ATTTAGAAAGTCACTATGACCATGT |
| | CRA2-R- | TGCTTTGTATTGTGAAGTTCGTCATCGCATAGATG |
| | CRA3 | ATTTAGAAACCCGGATTTTGACTGG |
| | CRA3-R- | CGAAATGTAGAAGATAGTTCGTCATCGCATAGATG |
| | BAP1 | ATTTAGAAACGGAATTCAAGGGGAC |
| | CRA1 RC | GAACTCAATCTGCATTATGCGAAGCAAGCGCGGA |
| | | GATTTC |
| | CRA2 RC | GAACTTCACAATACAAAGCAACATGGTCATAGTG |
| | | ACTTTC |
| | CRA3 RC | GAACTATCTTCTACATTTCGCCAGTCAAAATCCGG |
| | | GTTTC |
| PCR screening | Array screen F | GGAGTTCTGAGGTCATTACTGGATCTA |
| of arrays | | |
| | Array screen R | CAAATGTACGGCCAGCAACG |
| bap deletion | BAP 5′F | AGCAGCTGAGAGCCTGAATG |
| donor DNA | | |
| | BAP 5′R | ACATGCCAGCACTTAATCTGA |
| | BAP 3′F | TCAGATTAAGTGCTGGCATGTGCACCCAATCCCTA |
| | | ACATTAAACA |
| | BAP 3′R | GGTTCGGGCACCTCATCATT |
| CRAΦ deletion | CRA 5′F | ACAGGGCAGCCATTAACTGA |
| donor DNA | | |
| | CRA 5′R | TCTGAGACTGTAGCCTACGCA |
| | CRA 3′F | TGCGTAGGCTACAGTCTCAGAACGAAGTTATGTGC |
| | | CACAAGAAA |
| | CRA 3′R | TCAGACGCAAGCGTGAAGAT |
| bap deletion | BAP checkF | GCCTCCTAAAATTGGGGGCT |
| screening | | |
| | BAP checkR | CTTGGTTCTGCATTGGGTGC |
| CRAΦ deletion | CRA checkF | GACTTGCGTAGGCTTGGACT |
| screening | | |
| | CRA checkR | GCATGTCATGGTTTGGTGGG |
| | CRA circular | ATGAACGCGATCATTGCAGC |
| | F | |
| | CRA circular | TACGGCCAATTGATCACCCA |
| | R | |
| Cas12a/Cpf1 | GG-R12a- | TTTGGTCTCATAAGAACTTTAAATAATTTCTACTGT |
| CRISPR_{4xB1a} | Bla1 | TGTAGATCGGCGTCAATACGGGA |
| array | | |
| | Bla1-R12a- | TAATACCGCGCCACATGTCTAAGAACTTTAAATAA |
| | Bla2 | TTTCTACTGTTGTAGATGGAGCTGAATGAAGCC |
| | Bla2-R12a- | ATACCAAACGACGAGCGTCTAAGAACTTTAAATA |
| | Bla3 | ATTTCTACTGTTGTAGATCTCCCGTATCGTAGTT |
| | Bla3-R12a- | ATCTACACGACGGGGAGTCTAAGAACTTTAAATA |
| | Bla4 | ATTTCTACTGTTGTAGATAGCCGGAAGGGCCGAG |
| | Vector 3′F 12a | TTTGGTCTCAGTCTAAGAACTTTAAATAATTTCTAC |
| | GG | TGTTGTAGATCGGCCGGTAGAAAGGACA |
| | Vector 5′R 12a | TTTGGTCTCACTTAGACTAGAGTTAAGTCAAAACA |
| | GG | AAACCC |
| | Bla1 RC 12a | AGACATGTGGCGCGGTATTATCCCGTATTGACGCC |
| | | GATCT |
| | Bla2 RC 12a | AGACGCTCGTCGTTTGGTATGGCTTCATTCAGCTC |
| | | CATCT |
| | Bla3 RC 12a | AGACTCCCCGTCGTGTAGATAACTACGATACGGGA |
| | | GATCT |
| | Bla4 RC 12a | TTTGGTCTCAAGACCAGGACCACTTCTGCGCTCGG |
| | | CCCTTCCGGCTATCT |

TABLE 2
CRISPR spacers

| Name | Sequence |
|---|---|
| Kan1 Tl | GGTCGATCAGGGAGGATATCGGGGAAGAACAG |
| Kan1 T2 | GTCGATACTATGTTATACGCCAACTTTGAAAA |
| Kan1 B1 | TTGCATTCTAAAACCTTAAATACAGAAAACAG |
| Kan1 B2 | AAGCGAGCTCGGTACTAAAACAATTCATCCAG |
| Kan2 T1 | TCGCCGTCGGGCATGCGCGCCTTGAGCCTGGC |
| Kan2 T2 | CAACCTTACCAGAGGGCGCCCCAGCTGGCAAT |
| Kan2 B1 | GGCTACCTGCCCATTCGACCACCAAGCGAAAC |
| Kan2 B2 | GGCCGCTTGGGTGGAGAGGCTATTCGGCTATG |
| CRA1 | TCTCCGCGCTTGCTTCGCATAATGCAGATTGA |
| CRA2 | GTCACTATGACCATGTTGCTTTGTATTGTGAA |
| CRA3 | CCCGGATTTTGACTGGCGAAATGTAGAAGATA |
| BAP1 | CGGAATTCAAGGGGACAGGTAGCGCAGGTGAT |
| BAP2 | ATCGCGCGTTACCTCCTGAACATCCTCTACAG |
| BAP3 | GAGAAGTGAACTTGTCGATAAACCAATTTCAA |
| Random | TAGGGGAAAGCCTACTAGCCGGAGTGTTGCGA |

### DNA Sequence of Sample Genomically Integrating Vector, pp2.1-CRISPR_{8×Kan}-Spec-pp2.2

| LOCUS | |
|---|---|
| pp2.1-CR_4xAPH4x 3872 bp ss-DNA linear SYN 03 Jun. 2016 | |
| DEFINITION- | |
| ACCESSION- | |
| KEYWORDS- | |
| SOURCE- | |
| FEATURES | Location /Qualifiers |
| misc_feature | <1..1006 |
| | /note=“ADP1 prophage 2.1 region 2,158,257-2,159,574 [Split]” |
| misc_feature | 10007..1087 |
| | /note=“ ADP1 CRISPR upstream region” |
| primer_bind | complement(1064..1099) |
| | /note=“CRISPR 5′R 65” |
| primer_bind | 1072..1131 |
| | /note=“APH 5′-R-ST1” |
| repeat_region | 1088..1115 |
| | /note=“CR Repeat” |
| primer_bind | complement(1112..1151) |
| | /note=“APH ST1 RC” |
| primer_bind | 1132..1191 |
| | /note=“APH ST1-R-SB1” |
| repeat_region | 1148..1175 |
| | /note=“CR Repeat” |
| primer_bind | complement(1172..1211) |
| | /note=“APH SB1 RC” |
| primer_bind | 1192..1251 |
| | /note=“APH SB1-R-ST2” |
| repeat_region | 1208..1235 |
| | /note=“CR Repeat” |
| primer_bind | complement(1212..1231) |
| | /note=“Repeat RC” |
| primer_bind | complement(1232..1271) |
| | /note=“APH ST2 RC” |
| primer_bind | 1252..1311 |
| | /note=“APH ST2-R-SB2” |
| repeat_region | 1268..1295 |
| | /note=“CR Repeat” |
| primer_bind | complement(1292..1331) |
| | /note=“APH SB2 RC” |
| primer_bind | 1312..1371 |
| | /note=“APHB2-R-RCKT1” |
| repeat_region | 1328..1355 |
| | /note=“CR Repeat” |
| primer_bind | complement(1352..1391) |
| | /note=“RCK T1 RC” |
| primer_bind | 1372.1431 |
| | /note=“RCK T1-R-B1” |
| repeat_region | 1388..1415 |
| | /note=“CR Repeat” |
| primer_bind | complement(1412..1451) |
| | /note=“RCK B1 RC” |
| primer_bind | 1432..1491 |
| | /note=“RCK B1-R-T2” |
| repeat_region | 1448..1475 |
| | /note=“CR Repeat” |
| primer_bind | complement(1472..1511) |
| | /note=“RCK T2 RC” |
| primer_bind | 1492..1551 |
| | /note=“RCK T2-R-B2” |
| repeat_region | 1508..1535 |
| | /note=“CR Repeat” |
| primer_bind | complement(1532..1571) |
| | /note=“RCK B2 RC” |
| misc_feature | 1536..1567 |
| | /note=“RCK B2” |
| primer_bind | 1552..1611 |
| | /note=“RCK B2-R-spec” |
| repeat_region | 1568..1595 |
| | /note=“CR Repeat” |
| primer_bind | 1592..1611 |
| | /note=“Vector 3′F” |
| primer_bind | complement(1592..1611) |
| | /note=“ Array R” |
| promoter | 1656..1684 |
| | /note=“PampR” |
| CDS | 1719..2510 |
| | /codon_start=1 |
| | /db_xref=“GI:336359759” |
| | /gene=“specR” |
| | /note=“spectinomycin resistance marker” |
| | /product=“SpecR” |
| | /protein_id=“AEI53620.1” |
| | /transl_table=11 |
| | /translation=“MREAVIAEVSTQLSEVVGVIERHLEPTLLAVHLYGSAVDGGLKPH |
| | SDIDLLVTVTVRLDETTRRALINDLLETSASPGESEILRAVEVTIVVHDDIIPWRYPAK |
| | RELQFGEWQRNDILAGIFEPATIDIDLAILLTKAREHSVALVGPAAEELFDPVPEQDLF |
| | EALNETLTLWNSPPDWAGDERNVVLTLSRIWYSAVTGKIAPKDVAADWAMERLPAQYQP |
| | VILEARQAYLGQEEDRLASRADQLEEFVHYVKGEITKVVGK” |
| misc_feature | 2848..3872 |
| | /note=“ADP1 prophage 2.2 region 2,161,721-2,162,745” |
| primer_bind | complement(3852..3872) |
| | /note=“pp2.2 R63” |
| BASE COUNT | 1003 A 873 C 886 G 1110 T 0 OTHER |
| ORIGIN | ? |

```
      1 TGAGCCGACA TTTTATTACC CTCTTATCAA ACCGTACCTT TCACATAACG AATGAATGAA
    61 TACCGTACAT GGAGTGCGGC CAACCCACAG CGAACATCAT ATTTCGCATC CATCACCGTA
  121 CGGTTTTCCG TTTTAAGCTC TGCCCATGAT CTATCATGGA AATAACGGCT AATGATCACC
  181 TGCATCCACT CAAGTGTCGT TTCACTGTCT GTACCATTAA TAATATCCAG TACTAAACGT
  241 TGTACGGCAC GAGCTTCATT ATCGTTAATC TGACACGACA CTTTGTGACG TATAGCTTGT
  301 TGTACTCCTT GAGCATCACA AAGGTAATAA GCAATAAGTT TAGCTCGATC TTTCTTCTTT
  361 ACACGTACCT TGGCTTTCTT CATTGCAATA GCAATCGGGC TATCGGAATA CTGTCCACCA
  421 CGACAAGAAC GTTGCCACGC TCCACATTGG CGTAACCAAT CTGGCAAATC ATACTTGCTC
  481 CAATCCACCG TCTGCATAAT GTGCACTGCT GTATTCATCT CATCACCTAA TTTGTTTCAA
  541 GTTAAATTTT ATAAGCGTTA TTGTTTTATG GTTCTGCCTG CTCCTCTACC GATCTAAAAC
  601 GACAAGTTTC GAGATAATCC AGTACTCGAA CTGCACCGCG TTTACCGTGT CGGTTTTTCA
  661 CTACAATCAG CTCTGTGATT CCCATCGGTT TGGTTGAGTC TTCTGGATCG GTTAGGGGGT
  721 TAACAAGGAT GATCTGGTCT GCATCTTGCT CGATTTGTCC AGATTCTTTG ATATCTGATG
  781 CTTTAGGACG TTTGCCCTTC TCTGCCTCAC GGTTGAGCTG TACCAGTGCA ATGACAGGAC
  841 ATTCAAACTC TTTCGCCATG GATTTTAATT CACGGCTGAT GGAACTGACT TCCTGAAAGC
  901 GATCTTTCTT GCTCGGGTCT CTGAGCAGTT GTAAATAATC CACGATGATG CAGCCCAATT
  961 CCTTGTAACG GCGTTTGGCT CGACGTGCAT AGGAACGGAC CTCACTCAAG TGATTCATAA
1021 CGAAGTATTT TTACTCATTA AAAGCTTATA TAATTGATAT CAAGGGTTTT GTTTTGACTT
1081 AACTCTAGTT CGTCATCGCA TAGATGATTT AGAAAGGTCG ATCAGGGAGG ATATCGGGGA
1141 AGAACAGGTT CGTCATCGCA TAGATGATTT AGAAATTGCA TTCTAAAACC TTAAATACAG
1201 AAAACAGGTT CGTCATCGCA TAGATGATTT AGAAAGTCGA TACTATGTTA TACGCCAACT
1261 TTGAAAAGTT CGTCATCGCA TAGATGATTT AGAAAAAGCG AGCTCGGTAC TAAAACAATT
1321 CATCCAGGTT CGTCATCGCA TAGATGATTT AGAAATCGCC GTCGGGCATG CGCGCCTTGA
1381 GCCTGGCGTT CGTCATCGCA TAGATGATTT AGAAAGGCTA CCTGCCCATT CGACCACCAA
1441 GCGAAACGTT CGTCATCGCA TAGATGATTT AGAAACAACC TTACCAGAGG GCGCCCCAGC
1501 TGGCAATGTT CGTCATCGCA TAGATGATTT AGAAAGGCCG CTTGGGTGGA GAGGCTATTC
1561 GGCTATGGTT CGTCATCGCA TAGATGATTT AGAAACGGCC GGTAGAAAGG AGAAGCTTAC
1621 TAGCTATTTG TTTATTTTTC TAAATACATT CAAATATGTA TCCGCTCATG AGACAATAAC
1681 CCTGATAAAT GCTTCAATAA TATTGAAAAA GGAAGAGTAT GAGGGAAGCG GTGATCGCCG
1741 AAGTATCGAC TCAACTATCA GAGGTAGTTG GOGTCATCGA GCGCCATCTC GAACCGACGT
1801 TGCTGGCCGT ACATTTGTAC GGCTCCGCAG TGGATGGCGG CCTGAAGCCA CACAGTGATA
1861 TTGATTTGCT GGTTACGGTG ACCGTAAGGC TTGATGAAAC AACGCGGCGA GCTTTGATCA
1921 ACGACCTTTT GGAAACTTCG GCTTCCCCTG GAGAGAGCGA GATTCTCCGC GCTGTAGAAG
1981 TCACCATTGT TGTGCACGAC GACATCATTC CGTGGCGTTA TCCAGCTAAG CGCGAACTGC
2041 AATTTGGAGA ATGGCAGCGC AATGACATTC TTGCAGGTAT CTTCGAGCCA GCCACGATCG
2101 ACATTGATCT GGCTATCTTG CTGACAAAAG CAAGAGAACA TAGCGTTGCC TTGGTAGGTC
2161 CAGCGGCGGA GGAACTCTTT GATCCGGTTC CTGAACAGGA TCTATTTGAG GCGCTAAATG
2221 AAACCTTAAC GCTATGGAAC TCGCCGCCCG ACTGGGCTGG CGATGAGCGA AATGTAGTGC
2281 TTACGTTGTC CCGCATTTGG TACAGCGCAG TAACCGGCAA AATCGCGCCG AAGGATGTCG
2341 CTGCCGACTG GGCAATGGAG CGCCTGCCGG CCCAGTATCA GCCCGTCATA CTTGAAGCTA
2401 GACAGGCTTA TCTTGGACAA GAAGAAGATC GCTTGGCCTC GCGCGCAGAT CAGTTGGAAG
2461 AATTTGTCCA CTACGTGAAA GGCGAGATCA CCAAGGTAGT CGGCAAATAA TGTCTAACAA
2521 TTCGTTCAAG CCGAGGGGCC GCAAGATCCG GCCACGATGA CCCGGTCGTC GGTTCAGGGC
2581 AGGGTCGTTA AATAGCCGCT TATGTCTATT GCTGGTTTAC CGGTTTATTG ACTACCGGAA
2641 GCAGTGTGAC CGTGTGCTTC TCAAATGCCT GAGGTTTCAG CAAAAAACCC CTCAAGACCC
2701 GTTTAGAGGC CCCAAGGGGT TATGCTAGTT ATTGCTCAGC GGTGGCAGCA GCCTAGGTTA
2761 ATTAAGCTGC GCTAGTAGAC GAGTCCATGT GCTGGCGTTC AAATTTCGCA GCAGCGGTTT
2821 CTTTACCAGA CTCGACAAGC TTACTAGAGT GTTCATATTG ACCTCGCTTA GTGTGGTTAA
2881 TACGCCGCTT CTTGTTACTG CAAGAGGCGG TTTTTTTATG GGTGTACACA TGACTGCACC
2941 TGTTGATTCA GTTCAGATAG TGCTTTGTGC ATCTCATGGA TGACTCTGGC CATATCCAGT
3001 GCTTCGCCTT GGGTAATTCG CCCATCGGCC ATCATTTCTT TAAACAGTGC TGATATATCG
3061 CCCTTCTTGA TGCCTATGCA TAGGAAGGTA TCCATCAGAC TGGTATCTCG CTGGCTCTCG
3121 GGTATGTCTG GCAGGTCAAT TGCCACCTTT CCGAGTCGTG CACACATTTC CTGCAATATC
3181 CGATAGTCCC CTGTAATCTC CATCAGCTTG ACTGCCTCGA GCAATGTAAT GTGATGGGTA
3241 TGTGTGTTTG GGTTGACCTT GCTATTGAGC ACCGCAGGGC TTTTGATGCC TAAACGTGAT
3301 GCAAGTGCAG ATGCACCACC CAGAAAGTCG TGAACGGTGT GATAGGCAGC ATCTAATATG
3361 TTCATGGCGG GTTCCTTTGA ACGTGTTTAT TAGATGGGTG CTGACATAAG ATTGGATTTA
3421 TGGTTAGGAC GTAATTCAAT CCAAATATCT TCGTAATCAT CTGGAAATAA ATCTTTGCGA
3481 GTGCATAGAC CTTTATCTTC AGCAATTACA GCTAAGCGGA TTTTGCGATC TCTGGGAATT
3541 GCTTTCCATC CACTTACGGA TGCAGCGGTG ATACCTAAAA ATCTAGCAAC AGCAGTTACA
3601 CCACCTAAAA GCTCAATAAA TTGATCATCA GTCATGTTGA TCTCCTAATT TTATTGCCTC
3661 AATTATTAGG TATTCCTTAT ATTTTATCAA TAGGAATACC TTATTTATTT TATGTTAGGA
3721 TTTCCTAATA GACTAGGTAA GATCATGAAA ACATTAGCTG AACGACTTAA ATATGCGATG
3781 GAAATTTTGC CACCTAAGAA AATCAAGGGT GTCGAACTTG CTCGTGTAGT TGGAGTTAAA
3841 CCACCATCTG TCAGCGATTG GCTTTCAGGT AA
//
```

DNA Sequence of Sample Replicating Vector, pBAV1spec-CRISPR_{3×CRA}-Spec

| LOCUS pBAV1spec_CR_9xC 3162 bp DNA circular SYN 22 Mar. 2018 | |
|---|---|
| DEFINITION- | |
| ACCESSION- | |
| KEYWORDS- | |
| SOURCE- | |
| FEATURES | Location/Qualifiers |
| terminator | 81..158 |
| | /note=“t1” |
| CDS | complement(194..892) |
| | /codon_start=1 |
| | /db_xref=“GI:336359729” |
| | /gene=“repA” |
| | /note=“replication initiator protein” |
| | /product=“RepA” |
| | /protein_id=“AEI53594.1” |
| | /transl_table=11 |
| | /translation=“MAIKNTKARNFGFLLYPDSIPNDWKEKLESLGVSMAVSPLHDMDE |
| | KKDKDTWNSSDVIRNGKHYKKPHYHVIYIARNPVTIESVRNKIKRKLGNSSVAHVEILD |
| | YIKGSYEYLTHESKDAIAKNKHIYDKKDILNINDFDIDRYITLDESQKRELKNLLLDIV |
| | DDYNLVNTKDLMAFIRLRGAEFGILNTNDVKDIVSTNSSAFRLWFEGNYQCGYRASYAK |
| | VLDAETGEIK” |
| gene | complement(194..892) |
| | /gene=“repA” |
| CDS | complement(959..1120) |
| | /codon_start=1 |
| | /db_xref=“GI:336359730” |
| | /note=“ORFC” |
| | /product=“hypothetical protein” |
| | /protein_id=“AEI53595.1” |
| | /transl_table=11 |
| | /translation=“MVISESKKRVMISLTKEQDKKLTDMAKQKGFSKSAVAALAIEEYA |
| | RKESEQKK” |
| CDS | complement(1161..1370) |
| | /codon_start=1 |
| | /db_xref=“GI:336359731” |
| | /note=“ORFB” |
| | /product=“hypothetical protein” |
| | /protein_id=“AEI53596.1” |
| | /transl_table=11 |
| | /translation=“MGGKEANFASVLRPPIKCRVPIFVPKTLYPNWLKGLRGFSIANES |
| | PTFSPTFFINLYLSSFIVVFMITK” |
| repeat_region | 1323..1371 |
| | /note=“IRIII” |
| repeat_region | 1455..1477 |
| | /note=“IRII” |
| repeat_region | 1524..1655 |
| | /note=“IRI” |
| terminator | 1708..>1799 |
| | /note=“t0” |
| | primer_bind 1773..1799 |
| | /note=“Array screen F” |
| misc_feature | 1801..1880 |
| | /note=“CRISPR upstream reagion” |
| | primer_bind complement(1857..1892) |
| | /note=“Vector R” |
| | primer_bind 1865..1924 |
| | /note=“5′-R-CRA1” |
| repeat_region | 1881..1908 |
| | /note=“CR Repeat” |
| | primer_bind complement(1905..1944) |
| | /note=“CRA targ1 RC” |
| | primer_bind 1925..1984 |
| | /note=“CRA1-R-CRA2” |
| repeat_region | 1941..1968 |
| | /note=“CR Repeat” |
| | primer_bind complement(1965..2004) |
| | /note=“CRA targ2 RC” |
| | /note=“CRA2-R-CRA3” |
| repeat_region | 2001..2028 |
| | /note=“CR Repeat” |
| | primer_bind complement(2025..2064) |
| | /note=“CRA targ3 RC” |
| | primer_bind 2045..>2088 |
| | /note=“CRA3-R-PP1” |
| repeat_region | 2061..2088 |
| | /note=“CR Repeat” |
| | primer_bind complement(2075..2104) |
| | /note=“Array R” |
| primer_bind | 2085..2104 |
| | /note=“Vector F” |
| promoter | 2149..2177 |
| | /note=“PampR” |
| CDS | 2212..3003 |
| | /codon_start=1 |
| | /db_xref=“GI:336359759” |
| | /gene=“specR” |
| | /note=“spectinomycin resistance marker” |
| | /product=“SpecR” |
| | /protein_id=“AEI53620.1” |
| | /transl_table=11 |
| | /translation=“MREAVIAEVSTQLSEVVGVIERHLEPTLLAVHLYGSAVDGGLKPH |
| | SDIDLLVTVTVRLDETTRRALINDLLETSASPGESEILRAVEVTIVVHDDIIPWRYPAK |
| | RELQFGEWQRNDILAGIFEPATIDIDLAILLTKAREHSV ALVGPAAEELFDPVPEQDLF |
| | EALNETLTLWNSPPDWAGDERNVVLTLSRIWYSAVTGKIAPKDVAADWAMERLPAQYQP |
| | VILEARQAYLGQEEDRLASRADQLEEFVHYVKGEITKVVGK” |
| primer_bind | complement(2291..2310) |
| | /note=“Array screen R” |
| BASE COUNT | 899 A 688 C 627 G 948 T 0 OTHER |
| ORIGIN | ? |

```
      1 TAGAAAGGAG AAGCTTACTA GTAGCGGCCG CTGCAGGCCT CAGGGCCCGA TCGATGCCGC
    61 CGCTTAATTA ATTAATCCAG AGGCATCAAA TAAAACGAAA GGCTCAGTCG AAAGACTGGG
  121 CCTTTCGTTT TATCTGTTGT TTGTCGGTGA ACGCTCTCCT GAGTAGGACA AATCCGCCGC
  181 CCTAGACCTA GTGTCATTTT ATTTCCCCCG TTTCAGCATC AAGAACCTTT GCATAACTTG
  241 CTCTATATCC ACACTGATAA TTGCCCTCAA ACCATAATCT AAAGGCGCTA GAGTTTGTTG
  301 AAACAATATC TTTTACATCA TTCGTATTTA AAATTCCAAA CTCCGCTCCC CTAAGGCGAA
  361 TAAAAGCCAT TAAATCTTTT GTATTTACCA AATTATAGTC ATCCACTATA TCTAAGAGTA
  421 AATTCTTCAA TTCTCTTTTT TGGCTTTCAT CAAGTGTTAT ATAGCGGTCA ATATCAAAAT
  481 CATTAATGTT CAAAATATCT TTTTTGTCGT ATATATGTTT ATTCTTAGCA ATAGCGTCCT
  541 TTGATTCATG AGTCAAATAT TCATATGAAC CTTTGATATA ATCAAGTATC TCAACATGAG
  601 CAACTGAACT ATTCCCCAAT TTTCGCTTAA TCTTGTTCCT AACGCTTTCT ATTGTTACAG
  661 GATTTCGTGC AATATATATA ACGTGATAGT GTGGTTTTTT ATAGTGCTTT CCATTTCGTA
  721 TAACATCACT ACTATTCCAT GTATCTTTAT CTTTTTTTTC GTCCATATCG TGTAAAGGAC
  781 TGACAGCCAT AGATACGCCC AAACTCTCTA ATTTTTCCTT CCAATCATTA GGAATTGAGT
  841 CAGGATATAA TAAAAATCCA AAATTTCTAG CTTTAGTATT TTTAATAGCC ATGATATAAT
  901 TACCTTATCA AAAACAAGTA GCGAAAACTC GTATCCTTCT AAAAACGCGA GCTTTCGCTT
  961 ATTTTTTTTG TTCTGATTCC TTTCTTGCAT ATTCTTCTAT AGCTAACGCC GCAACCGCAG
1021 ATTTTGAAAA ACCTTTTTGT TTCGCCATAT CTGTTAATTT TTTATCTTGC TCTTTTGTCA
1081 GAGAAATCAT AACTCTTTTT TTCGATTCTG AAATCACCAT TTAAAAAACT CCAATCAAAT
1141 AATTTTATAA AGTTAGTGTA TCACTTTGTA ATCATAAAAA CAACAATAAA GCTACTTAAA
1201 TATAGATTTA TAAAAAACGT TGGCGAAAAC GTTGGCGATT CGTTGGCGAT TGAAAAACCC
1261 CTTAAACCCT TGAGCCAGTT GGGATAGAGC GTTTTTGGCA CAAAAATTGG CACTCGGCAC
1321 TTAATGGGGG GTCGTAGTAC GGAAGCAAAA TTCGCTTCCT TTCCCCCCAT TTTTTTCCAA
1381 ATTCCAAATT TTTTTCAAAA ATTTTCCAGC GCTACCGCTC GGCAAAATTG CAAGCAATTT
1441 TTAAAATCAA ACCCATGAGG GAATTTCATT CCCTCATACT CCCTTGAGCC TCCTCCAACC
1501 GAAATAGAAG GGCGCTGCGC TTATTATTTC ATTCAGTCAT CGGCTTTCAT AATCTAACAG
1561 ACAACATCTT CGCTGCAAAG CCACGCTACG CTCAAGGGCT TTTACGCTAC GATAACGCCT
1621 GTTTTAACGA TTATGCCGAT AACTAAACGA AATAAACGCT AAAACGTCTC AGAAACGATT
1681 TTGAGACGTT TTAATAAAAA ATCGCCTAGT GCTTGGATTC TCACCAATAA AAAACGCCCG
1741 GCGGCAACCG AGCGTTCTGA ACAAATCCAG ATGGAGTTCT GAGGTCATTA CTGGATCTAC
1801 AAGTGATTCA TAACGAAGTA TTTTTACTCA TTAAAAGCTT ATATAATTGA TATCAAGGGT
1861 TTTGTTTTGA CTTAACTCTA GTTCGTCATC GCATAGATGA TTTAGAAATC TCCGCGCTTG
1921 CTTCGCATAA TGCAGATTGA GTTCGTCATC GCATAGATGA TTTAGAAAGT CACTATGACC
1981 ATGTTGCTTT GTATTGTGAA GTTCGTCATC GCATAGATGA TTTAGAAACC CGGATTTTGA
2041 CTGGCGAAAT GTAGAAGATA GTTCGTCATC GCATAGATGA TTTAGAAACG GCCGGTAGAA
2101 AGGAGAAGCT TACTAGCTAT TTGTTTATTT TTCTAAATAC ATTCAAATAT GTATCCGCTC
2161 ATGAGACAAT AACCCTGATA AATGCTTCAA TAATATTGAA AAAGGAAGAG TATGAGGGAA
2221 GCGGTGATCG CCGAAGTATC GACTCAACTA TCAGAGGTAG TTGGCGTCAT CGAGCGCCAT
2281 CTCGAACCGA CGTTGCTGGC CGTACATTTG TACGGCTCCG CAGTGGATGG CGGCCTGAAG
2341 CCACACAGTG ATATTGATTT GCTGGTTACG GTGACCGTAA GGCTTGATGA AACAACGCGG
2401 CGAGCTTTGA TCAACGACCT TTTGGAAACT TCGGCTTCCC CTGGAGAGAG CGAGATTCTC
2461 CGCGCTGTAG AAGTCACCAT TGTTGTGCAC GACGACATCA TTCCGTGGCG TTATCCAGCT
2521 AAGCGCGAAC TGCAATTTGG AGAATGGCAG CGCAATGACA TTCTTGCAGG TATCTTCGAG
2581 CCAGCCACGA TCGACATTGA TCTGGCTATC TTGCTGACAA AAGCAAGAGA ACATAGCGTT
2641 GCCTTGGTAG GTCCAGCGGC GGAGGAACTC TTTGATCCGG TTCCTGAACA GGATCTATTT
2701 GAGGCGCTAA ATGAAACCTT AACGCTATGG AACTCGCCGC CCGACTGGGC TGGCGATGAG
2761 CGAAATGTAG TGCTTACGTT GTCCCGCATT TGGTACAGCG CAGTAACCGG CAAAATCGCG
2821 CCGAAGGATG TCGCTGCCGA CTGGGCAATG GAGCGCCTGC CGGCCCAGTA TCAGCCCGTC
2881 ATACTTGAAG CTAGACAGGC TTATCTTGGA CAAGAAGAAG ATCGCTTGGC CTCGCGCGCA
2941 GATCAGTTGG AAGAATTTGT CCACTACGTG AAAGGCGAGA TCACCAAGGT AGTCGGCAAA
3001 TAATGTCTAA CAATTCGTTC AAGCCGAGGG GCCGCAAGAT CCGGCCACGA TGACCCGGTC
3061 GTCGGTTCAG GGCAGGGTCG TTAAATAGCC GCTTATGTCT ATTGCTGGTT TACCGGTTTA
3121 TTGACTACCG GAAGCAGTGT GACCGTGTGC TTCTCAAATG CC
```

### Example 3 Rapid Assembly of Multiplex Natural CRISPR Arrays

Below is a non-limiting example of rapid assembly of multiplex natural CRISPR arrays as taught herein:

### Materials

1. DNA oligos for array assembly, as described in Methods. Standard quality desalted oligos in TE buffer or water have worked for us.
2. T4 DNA polynucleotide kinase.
3. T4 DNA ligase with buffer.
4. High-fidelity DNA polymerase (we used Q5 from New England Biolabs).
5. DpnI (if using a plasmid vector).
6. PCR tubes.
7. PCR thermocycler.
8. DNA electrophoresis machine for running gels.
9. PCR purification kit (we used Qiagen).
10. Gel purification kit (we used Qiagen).
11. Depending on your strategy for insertion into the vector, one of the following:
   a. BsaI or another Golden Gate Assembly-compatible restriction enzyme.
   b. Gibson Assembly master mix.
12. Vector template, which can be either a plasmid or linear DNA.
13. Competent cells.

### Methods

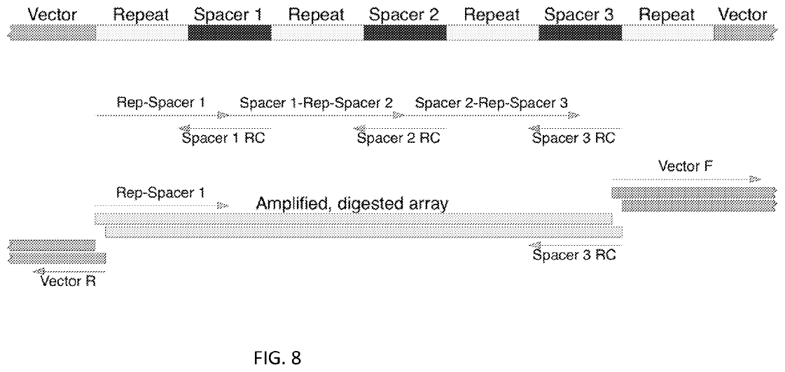

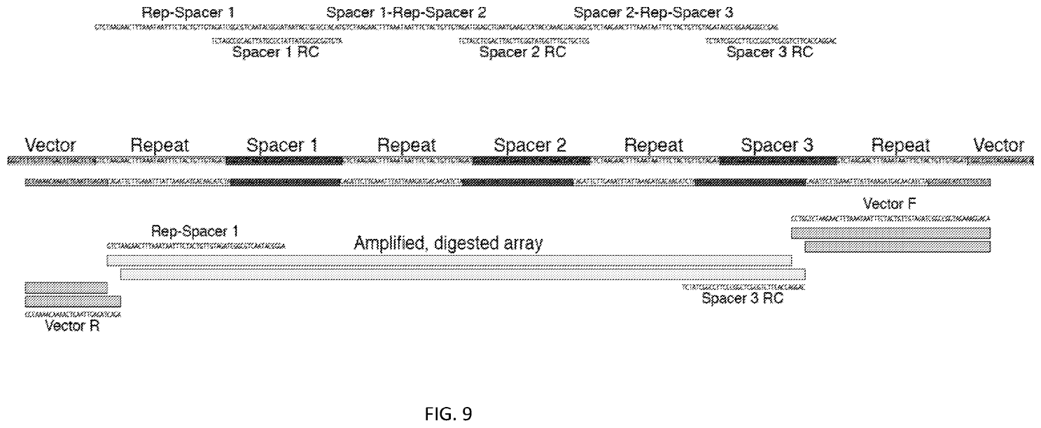

1. Prepare your vector. One vector compatible with a broad range of hosts that we have had success with is pBAV1k (Addgene #26702). For plasmids, PCR the plasmid with compatible Golden Gate adaptors (C. Engler, R. Kandzia, and S. Marillonnet (2008) A One Pot, One Step, Precision Cloning Method with High Throughput Capability, PLoS ONE. 3, e3647.). If using the restriction enzyme BsaI, append the Golden Gate adaptor sequence 5′-TTTGGTCTCA-3′ to the 5′ end of each primer (See Note 1). For the primer adjacent to the beginning of the array, after the Golden Gate adapter add the reverse complement of the first 4 bases of the CRISPR repeat. For the primer adjacent to the end of the array, add the last 4 bases of the final spacer and then the full CRISPR repeat, after the Golden Gate adapter and before the vector sequence (see Note 2, FIGS. 8, 9, and Table 3). Check your PCR on a gel, and if it looks good, purify it with a PCR purification kit. If the PCR product is significantly different in size from the parent plasmid, you can gel extract the product to separate it from the parent plasmid and reduce background when cloning.

2. Design oligos to use in assembling your CRISPR array (FIGS. 8, 9, Table 3). For an array of n spacers, you will need n top oligos and n bottom oligos. Bottom oligos should simply be the reverse complement of each spacer, followed by the reverse complement of the last 4 bases of the repeat at their 3′ ends (See Note 2). The bottom oligo for the final spacer in the array should also include a Golden Gate adaptor sequence at its 5′ end. All top oligos except the first should begin halfway through one spacer, span the repeat, and end halfway through the next spacer. The first top oligo should begin at the first repeat, end halfway through the first spacer, and include the Golden Gate adaptor at its 5′ end. Order standard desalted oligos and normalize to 100 μM in elution buffer, TE, or water.

3. Phosphorylate top oligos. Mix 1-2 μl of each top oligo (from 100 μM stock solutions), 1 μl T4 polynucleotide kinase, and T4 ligase buffer to 1×(See Note 3). Incubate at 37° C. for an hour. Alternatively, you could order 5′ phosphorylated top oligos.

4. Anneal oligos. Mix 2-6 μl of each bottom oligo, and then combine 1 part phosphorylated top oligos with 2-3 parts bottom oligos in a PCR tube. Heat to 85° C. in a thermocycler, and then slowly cool back to 37° C. at 0.1° C. per second (See Note 4).

5. Ligate oligos. Add 1 μl T4 DNA ligase and fresh T4 DNA ligase buffer to 1×. Incubate at 37° C. for about an hour. Leaving the ligation overnight is fine.

6. Remove unligated oligos. Purify the ligation using a PCR purification column.

7. Fill in the bottom strand and amplify. PCR the ligation using the first top oligo and final bottom oligo as primers. We used Q5 DNA polymerase, annealed at 72° C. (see Note 5), extended for 20 seconds, and ran for 20 cycles.

8. Purify the PCR product. For smaller, easier assemblies, purify the product using a PCR purification kit. For higher accuracy on difficult assemblies, instead run the ligation on a gel (after diluting to avoid overloading the wells), cut out the correct band, and purify the DNA using a gel extraction kit. If in doubt, run a test gel, and use gel extraction if the intended band is not the only clear product.

9. Insert the array into a vector. Combine 4 μl total of the vector and the PCR product at equimolar concentrations, 0.25 μl T4 DNA ligase, 0.25 μl BsaI, and 0.5 μl T4 DNA ligase buffer in a PCR tube. If your vector PCR came from a plasmid, also add 0.25 μl DpnI to cleave the parental plasmid. Incubate for 30-50 cycles of 1 minute each at 37° C. and 24° C., followed by 10 minutes at 50° C. to inactivate the enzymes. If you prefer to use Gibson Assembly (D.G. Gibson (2011) Enzymatic assembly of overlapping DNA fragments, Methods in enzymology. 498, 349-361.) to insert the array into your vector rather than a Golden Gate strategy, see Note 6.

10. If your vector is linear DNA, PCR amplify the final product.

11. Transform the product into your competent cells using a protocol appropriate for those cells, and grow clonal transformants.

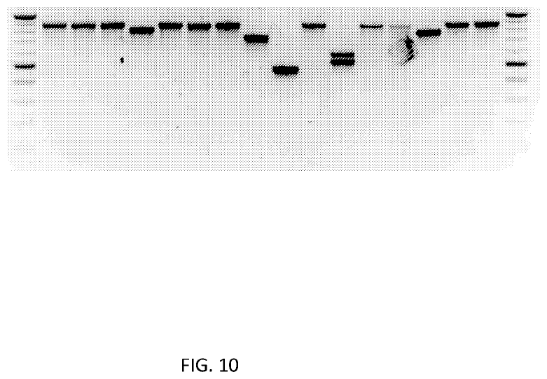

12. Pick several clones, extract their DNA using a protocol appropriate for your cells, and PCR and sequence across the array to verify correct assembly. For a representative screening PCR of clonal arrays, see FIG. 10. In this example, 11 of 16 clones had the correct number of spacers. Sequencing showed all of those 11 were assembled in the correct order. Seven of those were completely correct, and the remainder had small insertions, deletions, or substitutions.

### Notes

1. The Golden Gate adaptor sequence 5′-TTT GGTCTC A-3′ consists of 3 parts. The first three Ts simply extend the end of the DNA to help the restriction enzyme find its target site, and they could be replaced with any sequence. Here, we used BsaI with target site GGTCTC, but any other Golden Gate-compatible restriction enzyme would work as well. The final A is a spacer required because of the restriction enzyme's offset cutting site.

2. The exact end points of the assembled array are not critically important, so long as they provide unique ligation junctions for insertion into the vector. In the design provided here, the final repeat of the array is included in the vector PCR to reduce the length of the array to be assembled. The bottom oligo for the final spacer extends 4 bases into the repeat at its 3′ end to provide a 20-base annealing sequence for the primer in the PCR amplification step. Our spacers were 32 base pairs long, and only half of each spacer is included in the top oligo, so we added 4 bases to the bottom oligo to reach an annealing length of 20 base pairs (see FIGS. 8, 9). If your spacers are longer or shorter, you should adjust the extension of the bottom oligo into the repeat to ensure a 20 base annealing region for PCR. This is only important for the final spacer in the array, but we suggest ordering all bottom oligos with the same design to make them compatible with potential alternate array designs you may wish to assemble.

3. T4 polynucleotide kinase buffer generally omits ATP to allow users to supply their own radiolabeled version. T4 ligase buffer works as well and does not require additional ATP. Without ATP, the kinase will not work.

4. If your thermocycler cannot be programmed for a slow cooling step, you could heat a volume of water to near boiling, place the PCR tube containing the oligos in it, place it in a 37° C. water bath, and let it slowly come to equilibrium.

5. A high annealing temperature is critical for accurate amplification in this step. When using Q5, recommended annealing temperature for the primers can be checked using applicable software. If using another DNA polymerase, check the maximum allowed annealing temperature for your primers. Note also that using too many PCR cycles can make the PCR product less clean.

6. We have also successfully used Gibson Assembly to insert assembled arrays into their vectors. We find Golden Gate to be more accurate than Gibson Assembly in general, but both can work. The Gibson variation uses the same top strand-only ligation strategy to assemble the actual array; it just uses a different method to insert the array into a final vector. To use the Gibson method, you will need to prepare your vector differently in Methods Step 1, slightly change your oligo designs in Methods Step 2, and use a different vector insertion method in Methods Step 9.
a. In Methods Step 1, the forward primer for the vector (at the end of the CRISPR array) should begin just after the terminal CRISPR repeat in your final design. The reverse primer for the vector (at the beginning of the array) will begin just before the initial repeat. Depending on the length of the repeat units in your array, you can extend the primers slightly into the terminal repeats to ensure a 20-base overlap with the assembled array from Methods Step 8, for the final Gibson Assembly (see also below). Just be sure not to extend these overlaps so far into the repeats that the vector primers would anneal to each other.
b. In Methods Step 2, you will now need n+1 top oligos. The top oligo at the beginning of the array should begin 20 bases into the adjacent vector sequence, span the initial repeat, and end halfway through the first spacer. The top oligo at the end of the array should begin halfway through the last spacer, span the terminal repeat, and extend 20 bases into the adjacent vector sequence. The final bottom oligo should not include a Golden Gate adaptor sequence. If desired, you can reduce the top oligo overlaps with the vector sequence to avoid overly long oligos, and instead place the overlaps on the vector primers as described above.
c. In Methods Step 9, use Gibson Assembly to insert the assembled array into your vector. Combine 2 μl total of vector and array DNA at equimolar final concentrations in a PCR tube. Place in a thermocycler block preheated to 50° C. and add 2 μl of 2×Gibson Assembly master mix. Incubate at 50° C. for 1 hour.

FIG. 8 shows array assembly strategy for insertion into the vector using a Golden Gate approach. Top: A desired 3-spacer CRISPR array. Middle: 3 top and 3 bottom oligos to be used in assembling the array. Note that only the top strand is continuous after oligo annealing and ligation; the bottom strand has gaps at the repeats to ensure correct ligation junctions and spacer order. Golden Gate adaptors at the terminal oligos are not shown here. Bottom: PCR amplified, digested DNA pieces to be used for insertion of the CRISPR array into the vector using Golden Gate assembly, along with primers used to generate the pieces. Four-base 50 overlaps are shown at the junctions, which are created during Golden Gate assembly via digestion by BsaI or another compatible enzyme. In this scheme, the Golden Gate overlaps are at the first 4 bases of the repeat at the 5′ end, and the last 4 bases of the final spacer at the 3′ end.

TABLE 3
Oligos for assembling a sample 3-spacer
array for the Type I-F CRISPR-Cas system
of Acinetobacter baylyi (FIG. 9). Lower
case letters indicate Golden Gate assembly
adaptors, including a 5′ handle, the BsaI
recognition site GGTCTC, and a single base
spacer at the 3′ end. Italicized portions
indicate the repeat sequence.
RC denotes reverse complement.

| Category | Oligo | Sequence |
|---|---|---|
| Array Top | Repeat-Spacer 1 | tttggtctca- |
| | | GTCTAAGAACTTTA |
| | | AATAATTTCTACTG |
| | | TTGTAGAT-CGGCG |
| | | TCAATACGGGA |
| | Spacer 1- | TAATACCGCGCCACAT- |
| | Repeat-Spacer 2 | GTCTAAGAACTTTAA |
| | | ATAATTTCTACTGTT |
| | | GTAGAT-GGAGCTGA |
| | | ATGAAGCC |
| | Spacer 2- | ATACCAAACGACGAGC- |
| | Repeat-Spacer 3 | GTCTAAGAACTTTAA |
| | | ATAATTTCTACTGTT |
| | | GTAGAT-AGCCGGAA |
| | | GGGCCGAG |
| Array Bottom | Spacer 1 RC | ATGTGGCGCGGTAT |
| | | TATCCCGTATTG |
| | | ACGCCG-ATCT |
| | Spacer 2 RC | GCTCGTCGTTTGGT |
| | | ATGGCTTCATTC |
| | | AGCTCC-ATCT |
| | Spacer 3 RC | tttggtctca- |
| | | CAGGACCACTTCTG |
| | | CGCTCGGCCCTTCC |
| | | GGCT-ATCT |
| Vector | Vector F | tttggtctca-CCTG- |
| | | GTCTAAGAACTTTA |
| | | AATAATTTCTACTG |
| | | TTGTAGAT-CGGCC |
| | | GGTAGAAAGGACA |
| | Vector R | tttggtctca-AGAC- |
| | | TAGAGTTAAGTCAAA |
| | | ACAAAACCC |

### Additional Embodiments

Embodiment 1: A method of generating a CRISPR array, the method comprising:
providing a first oligonucleotide comprising a CRISPR repeat sequence, and a first portion of a first spacer sequence at its 3′ end;
providing a second oligonucleotide comprising, from 5′ to 3′, a second portion of the first spacer sequence, the CRISPR repeat sequence, and a first portion of a second spacer sequence;
providing a bridge oligonucleotide comprising, from 5′ to 3′, a sequence substantially complementary to a sequence at the 5′end of the CRISPR repeat sequence, a sequence substantially complementary to the first spacer sequence, and a sequence substantially complementary to a sequence at the 3′end of the CRISPR repeat sequence;
allowing the first oligonucleotide and the second oligonucleotide to hybridize with the bridge oligonucleotide; and
ligating the first and second oligonucleotide.

Embodiment 2. The method of Embodiment 1, wherein the first oligonucleotide further comprises, at its 5′ end, a portion of a flanking sequence.

Embodiment 3. The method of Embodiment 1, wherein the first oligonucleotide further comprises, at its 5′ end, a portion of a third spacer sequence.

Embodiment 4. The method of any one of Embodiments 1-3, wherein each of the first and second oligonucleotides comprises about 40 to about 70 nucleotides.

Embodiment 5. The method of Embodiment 4, wherein each of the first and second oligonucleotides comprises about 55 to about 65 nucleotides.

Embodiment 6. The method of any one of Embodiments 1-5, wherein the CRISPR repeat sequence comprises about 20 to about 36 nucleotides.

Embodiment 7. The method of any one of Embodiments 1-6, wherein the bridge oligonucleotide comprises about 30 to about 50 nucleotides.

Embodiment 8. The method of any one of Embodiments 1-7, wherein each of the first portion of the first spacer sequence, the second portion of the first spacer sequence, and the first portion of the second spacer sequence comprises about 12 to about 20 nucleotides.

Embodiment 9. The method of any one of Embodiments 1-8, wherein the sequence substantially complementary to a sequence at the 5′end of the CRISPR repeat sequence comprises about 3 to about 8 nucleotides.

Embodiment 10. The method of any one of Embodiments 1-9, wherein the sequence substantially complementary to a sequence at the 3′end of the CRISPR repeat sequence comprises about 3 to about 8 nucleotides.

Embodiment 11. The method of any one of Embodiments 1-10, wherein the first spacer sequence comprises a first target site in a target gene, and the second spacer sequence comprises a second target site in the target gene.

Embodiment 12. The method of any one of Embodiments 1-10, wherein the first spacer sequence comprises a target site in a first target gene, and the second spacer sequence comprises a target site in a second target gene.

Embodiment 13. The method of any one of Embodiments 1-12, wherein the bridge oligonucleotide is used at a ratio of between about 2:1 and about 3:1 by molarity in relation to a mixture of the first and second oligonucleotides.

Embodiment 14. The method of Embodiment 13, wherein the amount of the first and second oligonucleotides in the mixture are about equal.

Embodiment 15. The method of any one of Embodiments 1-14, comprising ligating three or more oligonucleotides.

Embodiment 16. The method of any one of Embodiments 1-15, wherein ligating the first and second oligonucleotides comprises using DNA ligase.

Embodiment 17. The method of any one of Embodiments 1-16, the method further comprises generating a strand complementary to the ligated first and second oligonucleotide, wherein the complementary strand comprises the bride oligonucleotide, thereby generating a double-strand construct.

Embodiment 18. The method of Embodiment 17, further comprising PCR amplification of the double-strand construct.

Embodiment 19. The method of Embodiment 18, further comprising inserting the PCR amplified construct into a vector.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of generating a CRISPR array, the method comprising:
providing a first oligonucleotide comprising a CRISPR repeat sequence or a portion thereof, and a first portion of a first spacer sequence at its 3′ end;
providing a second oligonucleotide comprising, from 5′ to 3′, a second portion of the first spacer sequence, the CRISPR repeat sequence, and a first portion of a second spacer sequence;
providing a bridge oligonucleotide comprising a sequence substantially complementary to the first spacer sequence;
allowing the first oligonucleotide and the second oligonucleotide to hybridize with the bridge oligonucleotide; and
ligating the first and second oligonucleotide.

2. The method of claim 1, wherein the first oligonucleotide further comprises, at its 5′ end, a flanking sequence.

3. The method of claim 2, wherein the first oligonucleotide comprises, from 5′ to 3′, a flanking sequence, a CRISPR repeat sequence or a portion thereof, and a first portion of a first spacer sequence.

4. The method of claim 3, wherein the flanking sequence comprises a portion of a sequence of a vector.

5. The method of claim 1, wherein the first oligonucleotide further comprises, at its 5′ end, a portion of a third spacer sequence.

6. The method of claim 5, wherein the first oligonucleotide comprises, from 5′ to 3′, a portion of a third spacer sequence, a CRISPR repeat sequence or a portion thereof, and a first portion of a first spacer sequence.

7. The method of claim 6, wherein the bridge oligonucleotide further comprises a sequence substantially complementary to a portion of the CRISPR repeat sequence at its 5′ or 3′ end.

8. The method of claim 7, wherein the portion of the CRISPR repeat sequence comprises about 1 to about 10 nucleotides.

9. The method of claim 7, wherein the bridge oligonucleotide comprises, from 5′ to 3′, a sequence substantially to a first portion of the CRISPR repeat sequence, the sequence substantially complementary to the first spacer sequence, and a sequence substantially complementary to a second portion of the CRISPR repeat sequence.

10. The method of claim 9, wherein the first and/or second portion of the CRISPR repeat sequence comprises about 1 to about 10 nucleotides.

11. The method of any one of claim 1, wherein each of the first and second oligonucleotides comprises about 40 to about 70 nucleotides.

12. The method of claim 11, wherein each of the first and second oligonucleotides comprises about 55 to about 65 nucleotides.

13. The method of claim 1, wherein the CRISPR repeat sequence comprises about 15 to about 36 nucleotides.

14. The method of claim 9, wherein the bridge oligonucleotide comprises about 30 to about 50 nucleotides.

15. The method of claim 1, wherein each of the first portion of the first spacer sequence, the second portion of the first spacer sequence, and the first portion of the second spacer sequence comprises about 5 to about 20 nucleotides.

16. The method of claim 15, wherein the first spacer sequence comprises a first target site in a target gene, and the second spacer sequence comprises a second target site in the target gene.

17. The method of claim 15, wherein the first spacer sequence comprises a target site in a first target gene, and the second spacer sequence comprises a target site in a second target gene.

18. The method of claim 14, wherein the bridge oligonucleotide is used at a ratio of between about 2:1 and about 3:1 by molarity in relation to a mixture of the first and second oligonucleotides.

19. The method of claim 18, wherein the amount of the first and second oligonucleotides in the mixture are about equal.

20. The method of claim 1, wherein the first oligonucleotide, the second oligonucleotide, and the bridge oligonucleotide are DNA oligonucleotides.

21. (canceled)

22. (canceled)

23. (canceled)

24. (canceled)

25. The method of claim 1, the method further comprises generating a strand complementary to the ligated first and second oligonucleotide, wherein the complementary strand comprises the bride oligonucleotide, thereby generating a double-strand construct.

26. (canceled)

27. (canceled)
